SEQUENCE LISTING (80 sequences)

SEQ ID NO: 1 - DNA, 51 bp, Artificial Sequence
  Primer
aagcagtggtaacaacgcagagtgaattaatacgactcactatagggagat

SEQ ID NO: 2 - DNA, 30 bp, Artificial Sequence
  Primer
aagcagtggtaacaacgcagagtacgcggg

SEQ ID NO: 3 - DNA, 23 bp, Artificial Sequence
  Primer
aagcagtggtaacaacgcagagt

SEQ ID NO: 4 - DNA, 25 bp, Artificial Sequence
  Primer
tggatgacatctacaaggctgcggt

SEQ ID NO: 5 - DNA, 25 bp, Artificial Sequence
  Primer
gagttcaaggcagccttcgacatct

SEQ ID NO: 6 - DNA, 25 bp, Artificial Sequence
  Primer
atggctgcatcagcaccaaggagct

SEQ ID NO: 7 - DNA, 25 bp, Artificial Sequence
  Primer
ggtggactttgatgagttcctggtc

SEQ ID NO: 8 - DNA, 25 bp, Artificial Sequence
  Primer
gttcctggtcatgatggttcggtgc

SEQ ID NO: 9 - DNA, 25 bp, Artificial Sequence
  Primer
ggttcggtgcatgaaggacgacagc

SEQ ID NO: 10 - DNA, 25 bp, Artificial Sequence
  Primer
gggaaatctgaggagctgtctgacc

SEQ ID NO: 11 - DNA, 25 bp, Artificial Sequence
  Primer
aatgctgcaggctacaggcgagacc

SEQ ID NO: 12 - DNA, 25 bp, Artificial Sequence
  Primer
tacaggcgagaccatcacggaggac

SEQ ID NO: 13 - DNA, 25 bp, Artificial Sequence
  Primer
ggaggacgacatcgaggagctcatg

SEQ ID NO: 14 - DNA, 25 bp, Artificial Sequence
  Primer
gacggccgcatcgactatgatgagt

SEQ ID NO: 15 - DNA, 26 bp, Artificial Sequence
  Primer
cgcagagtgaattaatacgactcact

SEQ ID NO: 16 - DNA, 25 bp, Artificial Sequence
  Primer
ggcagtgatgggtgcttggttccaa

SEQ ID NO: 17 - DNA, 25 bp, Artificial Sequence
  Primer
acaggactcgtcaagcttctattac

SEQ ID NO: 18 - DNA, 25 bp, Artificial Sequence
  Probe
ggcgacatggccaaacgtaccaaga

SEQ ID NO: 19 - DNA, 25 bp, Artificial Sequence
  Probe
gtaccaagaaagtcgggatcgtcgg

SEQ ID NO: 20 - DNA, 25 bp, Artificial Sequence
  Probe
aattgaaatcagccagcacgccaag

SEQ ID NO: 21 - DNA, 25 bp, Artificial Sequence
  Probe
tggcactgtggttcctgcatgaaga

SEQ ID NO: 22 - DNA, 25 bp, Artificial Sequence
  Probe
gcactgtggttcctgcatgaagaca

SEQ ID NO: 23 - DNA, 25 bp, Artificial Sequence
  Probe
tgcatgaagacagtggctggcggtg

SEQ ID NO: 24 - DNA, 25 bp, Artificial Sequence
  Probe
ccacttccgctgtcacggtaaagtc

SEQ ID NO: 25 - DNA, 25 bp, Artificial Sequence
  Probe
tccgctgtcacggtaaagtccgcca

SEQ ID NO: 26 - DNA, 25 bp, Artificial Sequence
  Probe
gctgtcacggtaaagtccgccatca

SEQ ID NO: 27 - DNA, 25 bp, Artificial Sequence
  Probe
gtcacggtaaagtccgccatcagaa

SEQ ID NO: 28 - DNA, 25 bp, Artificial Sequence
  Probe
ggtaaagtccgccatcagaagactg

SEQ ID NO: 29 - DNA, 30 bp, Cattle
gttgcgttaacatggctaaacgcaccaaga

SEQ ID NO: 30 - DNA, 25 bp, Cattle
gcaccaagaaggtcggaatcgtggg

SEQ ID NO: 31 - DNA, 25 bp, Cattle
aattgaaatcagccagcacgccaag

SEQ ID NO: 32 - DNA, 25 bp, Cattle
tggcactgtggttcctgcatgaaaa

SEQ ID NO: 33 - DNA, 25 bp, Cattle
gcactgtggttcctgcatgaaaaca

SEQ ID NO: 34 - DNA, 25 bp, Cattle
tgcatgaaaacagtagctggtggtg

SEQ ID NO: 35 - DNA, 25 bp, Cattle
ccacctctgccgtcacagtcaagtc

SEQ ID NO: 36 - DNA, 25 bp, Cattle
tctgccgtcacagtcaagtccgcca

SEQ ID NO: 37 - DNA, 25 bp, Cattle
gccgtcacagtcaagtccgccatca

SEQ ID NO: 38 - DNA, 25 bp, Cattle
gtcacagtcaagtccgccatcagaa

SEQ ID NO: 39 - DNA, 25 bp, Cattle
agtcaagtccgccatcagaagactg

SEQ ID NO: 40 - DNA, 25 bp, Cattle
tggatgacatctacaaggctgcggt

SEQ ID NO: 41 - DNA, 25 bp, Cattle
gagttcaaggcggcctttgacatct

SEQ ID NO: 42 - DNA, 25 bp, Cattle
atggctgcatcagcaccaaggagct

SEQ ID NO: 43 - DNA, 25 bp, Cattle
agtggactttgatgagttcttggtc

SEQ ID NO: 44 - DNA, 25 bp, Cattle
gttcttggtcatgatggttcggtgc

SEQ ID NO: 45 - DNA, 25 bp, Cattle
ggttcggtgcatgaaggatgacagc

SEQ ID NO: 46 - DNA, 29 bp, Cattle
aggaaagtctgaggaagagctttcagacc

SEQ ID NO: 47 - DNA, 25 bp, Cattle
aatgcttcaggctacaggggagacc

SEQ ID NO: 48 - DNA, 25 bp, Cattle
tacaggggagaccatcacagaggac

SEQ ID NO: 49 - DNA, 25 bp, Cattle
agaggacgacattgaggagctcatg

SEQ ID NO: 50 - DNA, 25 bp, Cattle
gatggccgcatcgactatgatgagt

SEQ ID NO: 51 - DNA, 25 bp, Artificial Sequence
  Probe
tggcctctgcccgagcttcagaggc

SEQ ID NO: 52 - DNA, 25 bp, Artificial Sequence
  Probe
tgcccgagcttcagaggccgaggat

SEQ ID NO: 53 - DNA, 25 bp, Artificial Sequence
  Probe
ttcagaggccgaggatgcctccctt

SEQ ID NO: 54 - DNA, 25 bp, Artificial Sequence
  Probe
caccaagaccgccaaggatgcactg

SEQ ID NO: 55 - DNA, 25 bp, Artificial Sequence
  Probe
gaccgccaaggatgcactgagcagc

SEQ ID NO: 56 - DNA, 25 bp, Artificial Sequence
  Probe
aggatgcactgagcagcgtgcagga

SEQ ID NO: 57 - DNA, 25 bp, Artificial Sequence
  Probe
ccgatggcttcagttccctgaaaga

SEQ ID NO: 58 - DNA, 25 bp, Artificial Sequence
  Probe
ccctgaaagactactggagcaccgt

SEQ ID NO: 59 - DNA, 25 bp, Artificial Sequence
  Probe
gactactggagcaccgttaaggaca

SEQ ID NO: 60 - DNA, 25 bp, Artificial Sequence
  Probe
gcaccgttaaggacaagttctctga

SEQ ID NO: 61 - DNA, 25 bp, Artificial Sequence
  Probe
ggaccctgaggtcagaccaacttca

SEQ ID NO: 62 - DNA, 32 bp, Dog
tggcctccgcccgagccctggaggaagaggac

SEQ ID NO: 63 - DNA, 25 bp, Dog
tgaccagcgttcaggagtcccaggt

SEQ ID NO: 64 - DNA, 25 bp, Dog
tggaggaagaggacccctccctcct

SEQ ID NO: 65 - DNA, 25 bp, Dog
caccaagacggcccaggacacgctg

SEQ ID NO: 66 - DNA, 25 bp, Dog
gacggcccaggacacgctgaccagc

SEQ ID NO: 67 - DNA, 25 bp, Dog
aggacacgctgaccagcgttcagga

SEQ ID NO: 68 - DNA, 25 bp, Dog
ccgatagcttcagttccctgaaaga

SEQ ID NO: 69 - DNA, 24 bp, Dog
ccctgaaagactactgcagcacgt

SEQ ID NO: 70 - DNA, 25 bp, Dog
gactactgcagcacgtttaagggca

SEQ ID NO: 71 - DNA, 25 bp, Dog
gcacgtttaagggcaagttcactgg

SEQ ID NO: 72 - DNA, 31 bp, Dog
ggattcagcctctgaggccaaaccaactcca

SEQ ID NO: 73 - DNA, 237 bp, Napa
  misc_feature 196: n = A,T,C or G
atctactggggactggtcctattgatacctttttgcacatcttgctgatcctggccatgccactatcttcgctgctttcacacccaagtggagacgtcaagaaaggcaggatttgggagggagaatgcatttgtgtgatcatgtttgtacaaatatggtttttgaacttaaaatattatgtatttatacatattgnggtcgaatgcaaattagggtttcgcgtcaaaaaaaaaaaaa

SEQ ID NO: 74 - DNA, 225 bp, Brassica rapa
cccatctactggggactggtcctattgataacctttttgcacatcttgctgatcctggccatgccactatcttcgctgctttcacacccaagtgagacgtcaagaaaggaggatttgggagggagaatgcatttgtgtgatcatgtttgtacaaatatggtttttgaacttaaaatattatgtatttatacatattgtggtcgaatgcaaattagggtttcgcgg

SEQ ID NO: 75 - DNA, 271 bp, Cauliflower
ccatctactgggactggtcctattgatacctttttgcacatcttgctgatcctggccatgctactatcttcgctgctttcacacccaagtgagacgtcaagaaaggaggatttgggagggataatgcgtttgtgtgatcatgtttgtacaaatgtggtttttgaacttaaaaatattatgtatttaagcaaatggttgtcgaatgcaagttagggttcgccgtaaacactgctcttttcctgactaactgctcatacattgtggttcgtgg

SEQ ID NO: 76 - DNA, 234 bp, Arabidopsis thaliana
tctcctggcctggtcctattgatacctttttgcacatcttgctgatcctggtcacgccacaatcttcgctgctttcacacccaagtgagacaacgaaaaagggttatggggaggtgaatgcgtttgtgtgtgatcatgtttgtacaaatatcattttgaacttaaaattattatgtactttatataaattgtggtcgaatgcaaattaaggttccccgtaaaaaaaaaaaaaaa

SEQ ID NO: 77 - DNA, 257 bp, Napa
gagctatcgagattatctctgggagcttctgcacttgaagccgctaaataatcacatcctcgttgttttgcgttaaacttggcttttgatatatctatccaccgtaaaggagaaagagtgtggtttcaactcttaaaagactactgaaatcaattttcttttgctggcttcttgttgagacaaccagatttggctaagatgtttcttgtaataaatttccagcaggataacgaatattcaccttttgcaaacaaaaa

SEQ ID NO: 78 - DNA, 216 bp, Brassica rapa
  misc_feature 39: n = A,T,C or G
acgaatattcaccttttgcaaacaaaaacgttggcgtgngtatgaacttggcattttgatatgtctatccaccgttttggagaaagagtgtgggttcactgcttgacagactactgaaatgcattttcttttgctggcttcttgttgagacaaccagctttggctaagatgtttcttgtaataaatttccctgcgatttccggccggataacgaac

SEQ ID NO: 79 - DNA, 278 bp, Arabidopsis thaliana
ctattgagattatctctgggagcttctgctcttgaagctgctaaataagcccatccgtgttagttctcttttcttcgctttgatatttacccccaccttgaaggagaagagtgtgttaaaattttactgaatcagtttccttgctggcttcttgttgagacctaccaatgtttgctcaacgatttttaataaacatacctgtaatttccagcaggatttcgaataattgcattaacattttgttctgattcagactcgcttcctccagagtctgtagt

SEQ ID NO: 80 - DNA, 183 bp, Cauliflower
agctatcgagattatctctgggagcttctgcacttgaagctgcaaataagcacatccttgtctgagttatgatatctatccaccttgaaggagaggagtctgttttgaacaacctttgctggcttcttgttggcaccaccagtgtttgcccaaaagattctttttttttttttaaaaaaaaaa

(19) United States
(12) Patent Application Publication
(10) Pub. No.: US 2008/0268437 A1
(43) Pub. Date: Oct. 30, 2008

(54) Method of Targeted and Comprehensive Sequencing Using High-Density Oligonucleotide Array

(76) Inventors: Wan Ji, Cary, NC (US); Wenli Zhou, Austin, TX (US); Sara F. Davis, Cedar Park, TX (US); Scott K. Davis, Bertram, TX (US)

Correspondence Address: MORRISON & FOERSTER LLP, 425 MARKET STREET, SAN FRANCISCO, CA, 94105-2482, US

(21) Appl. No.: 11/664,023

(22) PCT Filed: Sep. 27, 2005
(86) PCT No.: PCT/US05/35292

§ 371 Date: Mar. 21, 2008

Related U.S. Application Data
(60) Provisional application No. 60/614,003, filed on Sep. 27, 2004.

(51) Int. Cl. C12Q 1/68; C40B 20/04; C12P 19/34

(52) U.S. Cl. 435/6; 506/4; 435/91.2

(57) ABSTRACT

A method for targeted and comprehensive sequencing using high-density oligonucleotide array, comprising the steps of hybridizing nucleic acid from an investigative species with high-density oligonucleotide arrays of a related species, identifying the oligonucleotide probes that generate high hybridization signals, using the probes sequences to make PCR primers, amplifying heterologous genes by PCR with the gene specific PCR primers and an anchoring primer, and sequencing the PCR products.

### RELATED APPLICATIONS

This application claims the benefit of U.S. Provisional Applications No. 60/614,003 filed Sep. 27, 2004, which is incorporated by reference herein in its entirety.

### TECHNICAL FIELD OF THE INVENTION

The present invention discloses a method of extracting broad and extensive genomic sequence information from an organism in a general, rapid, and directed manner.

### BACKGROUND OF THE INVENTION

It is well established that the phenotypic characteristics of an organism are controlled by the interplay of its genetic components with the environment in which the organism lives. To understand the biological development or the pathological derailment of such organism, it is essential to dissect and analyze the genetic components which consist of long DNA sequences made up of four different nucleotides. The methods of DNA sequencing and its industrial automations have long been established (see Proc Natl Acad Sci USA. 74:5463 (1977), Clin Chem. 35:2196 (1989)), which culminated in the recent completion of draft human genome (see Science. 291:1304 (2001), Nature. 409:860 (2001)). Despite this monumental achievement, large scale sequencing is still expensive and time-consuming, entailing large investments in lab equipment and computing infrastructure, which limits its application to a wide range of agriculture, aquaculture and wildlife research.

The draft human genome has provided us a panoramic view of its constituents. However, in spite of the complexity of human beings, our underlying genome has only about 35,000 genes (see Science. 291:1304 (2001), Nature. 409:860 (2001)). It is the specific patterns of expression of these genes, both spatially and temporally, that determines human biology. Therefore, the most productive way to understand the biology of an organism is to sequence its functional genes and their surrounding regulatory segments. The task, however, is greatly complicated and enormously increased by the disparity between the expression of different genes. Typically, a few dominantly expressed genes, which are related to the physiological functions of the particular cell-type, constitute a vast majority of the RNA in such cell. For example, a few myosin genes and a few lipoprotein genes constitute more than 90% of all RNA in heart and liver, respectively. Thus, using the most popular shotgun sequencing strategy, by which expressed genes are randomly selected for sequencing, some genes will be wastefully interrogated again and again while other important ones are ignored. Statistically, only through costly large-scale sequencing can a comprehensive coverage of expressed genes be achieved. Several methods have been developed to increase the efficiency of sequencing efforts targeted to expressed genes and the most widely used method creates a normalized cDNA library (see Proc Natl Acad Sci USA. 91:9228 (1994)). The process is, however, quite complex and requires specially trained personnel. Moreover, although the disparity among expressed genes to certain extent, the so called “normalized” cDNA library is not truly normalized. As a result, the shotgun method is still the most popular choice in large scale cDNA sequencing, despite its huge wastefulness.

Another problem in large scale cDNA sequencing methods is the complete lacking of prior knowledge of the genes being sequenced. The function of a cDNA sequence can only be inferred afterward by comparing with genes of similar sequence in other species. It is not difficult to manually annotate a few cDNAs. It is, however, a considerable effort to annotate hundreds and thousands of cDNAs generated by large scale sequencing, requiring experienced bioinformaticians and computer infrastructure not found in average labs.

There remains a need for a rapid, efficient method to generate sequence data for genes of interest from organisms lacking extensive, public sequence databases.

### SUMMARY OF THE INVENTION

This invention addresses these needs by providing a method to extract genomic and expressed gene sequences from different organisms in a general, rapid, and directed manner. In particular, this invention provides a method to identify probes or PCR primers that can be used to amplify and/or clone homologues and related genes from different organisms on a large scale and in a rapid and directed manner. Transcript sequences of a species of interest are simultaneously hybridized to microarrays containing immobilized nucleic acids from another species. Hybridization of transcript sequences from the species of interest to oligonucleotides on a microarray from another species provides information on the appropriate probes to use for library screening or on the appropriate PCR primers to use to amplify the corresponding sequence from the species of interest that served as the source of the transcript sequences. The transcript from the species of interest can be amplified with the target specific primer(s) identified from the microarray and a universal anchoring primer.

In an embodiment, the invention provides a method of identifying and selecting a primer suitable for PCR amplification of a heterologous nucleic acid by synthesizing a nucleic acid corresponding to an expressed gene sequence from a species of interest, hybridizing the nucleic acid to a microarray of oligonucleotides derived from expressed gene sequences of a species different from the species of interest, detecting hybridization of the nucleic acid to one or more oligonucleotides on the microarray, and then identifying and selecting the one or more oligonucleotides which have hybridized to the nucleic acid to levels suitable to act as PCR primers. Additionally, the invention provides methods for using the identified and selected oligonucleotides to perform PCR amplification, sequencing, and cloning of heterologous genes of interest.

### DESCRIPTION OF THE DRAWINGS

FIG. 1 shows GeneChip Human Genome U133A hybridization images of ribosomal protein L37a from heart and liver of human, cattle, dog, and pig. PM and MM denote perfect match probes and mismatch probes respectively. The probes that produce a relatively high hybridization signal are labeled with a star.

FIG. 2 shows GeneChip Human Genome U133A hybridization images of troponin C from heart and liver of human, cattle, dog, and pig. PM and MM denote perfect match probes and mismatch probes respectively. The probes that produce a relatively high hybridization signal are labeled with a star.

FIG. 3 shows GeneChip Human Genome U133A hybridization images of apolipoprotein C-III from heart and liver of human, cattle, dog, and pig. PM and MM denote perfect match probes and mismatch probes, respectively. The probes that produce a relatively high hybridization signal are labeled with a star.

FIG. 4 shows a sequence comparison of GeneChip probes and their heterologous targets. The mutated nucleotides are labeled with bold, italic and underlined fonts. The probes that produced a relatively high hybridization signal in the heterologous hybridization experiments (FIGS. 1-3) are labeled with a star.

FIG. 5 shows an image of agarose-gel electrophoresis of the PCR products of human troponin C. It demonstrates PCR amplification of troponin C from human heart using Affymetrix human GeneChip probes. Hybridization signals of the 11 GeneChip probes are labeled under each respective lane.

FIG. 6 shows an image of agarose-gel electrophoresis of the PCR products of cattle Troponin C. It demonstrates PCR amplification of troponin C from cattle heart using Affymetrix human GeneChip probes. Hybridization signals of the 11 GeneChip probes are labeled under each respective lane.

FIG. 7 shows an image of agarose-gel electrophoresis of the PCR products of dog Troponin C. It demonstrates PCR amplification of troponin C from dog heart using Affymetrix human GeneChip probes. Hybridization signals of the 11 GeneChip probes are labeled under each respective lane.

FIG. 8 is GeneChip Arobidopsis ATH1 hybridization images of chlorophyll A-B binding protein of Arabodopsis thaliana, Cauliflower, Napa and Brassica rapa. PM and MM denote perfect match probes and mismatch probes respectively. The probes that produce a relatively high hybridization signal are labeled with a star.

FIG. 9 is GeneChip Arobidopsis ATH1 hybridization images of mitochondrial F1-ATPase, gamma subunit of Arabodopsis thaliana, Cauliflower, Napa and Brassica rapa. PM and MM denote perfect match probes and mismatch probes respectively. The probes that produce a relatively high hybridization signal are labeled with a star. Amplification of chlorophyll A-B binding protein of Arabodopsis thaliana, Cauliflower, Napa and Brassica rapa.

FIG. 10 is an image of agarose-gel electrophoresis of the PCR products of plant chlorophyll A-B binding protein.

FIG. 11 is an image of agarose-gel electrophoresis of the PCR products of plant mitochondrial F1-ATPase, gamma subunit.

FIG. 12 shows the chlorophyll A-B binding protein sequences obtained from above sequencing reaction. All four sequences are highly homology to each other. Sequence alignment reveals highly conserved regions (marked as stars) as well as divergent regions.

FIG. 13 shows the F1-ATPase sequences obtained from above sequencing reaction. The conserved nucleotides are marked with stars.

### DESCRIPTION OF PREFERRED EMBODIMENTS OF THE INVENTION

Although various features of the invention are described herein, it is to be understood that the present invention is not limited to the particular embodiments described in this disclosure. It will be clear to one of skill in the art that the present invention can be practiced or carried out in various ways and can be have additional embodiments. The terminology used in this disclosure is meant to describe the invention and is not to be regarded as limiting.

In the disclosure of this invention, various patents, published patent applications, and publications are cited. The disclosures from these sources are hereby incorporated by reference into the present disclosure to provide a description of the field and state of the art to which this invention pertains. The practice of this invention will employ, unless noted otherwise, conventional methods in biochemistry, molecular biology, and recombinant DNA, among others, which are well known to one of skill in the art. Technical terms are used according to their conventional usage.

### DEFINITIONS

As used herein, the term “heterologous” can refer to related nucleic acid molecules derived from different species. Accordingly, a heterologous nucleic acid can represent a homologue, orthologue, or related gene in another species of interest. A heterologous nucleic acid can have 50%, 55%, 60%, 65%, 70%, 75%, 80%, 85%, 90%, or 95% or more percent sequence identity to a gene of interest. As an alternative to identity, a heterologous nucleic acid may be defined by its ability to hybridize to a gene of interest under low, moderate, high or very high stringency hybridization and wash conditions. As used herein, the phrase “low stringency conditions” refers the following conditions and equivalents thereto: hybridization at 5× SSC, 2% SDS, and 100 μg/ml single stranded DNA at 40° C. for 16 hours, followed by washing in 2× SSC, 0.2% SDS, at 50° C. for thirty minutes.

As used herein, the phrase “moderate stringency conditions” refers the following conditions and equivalents thereto: hybridization at 5× SSC, 2% SDS, and 100 μg/ml single stranded DNA at 50° C. for 16 hours, followed by washing in 0.2× SSC, 0.2% SDS, at 50° C. for thirty minutes.

As used herein, the phrase “high stringency conditions” refers the following conditions and equivalents thereto: hybridization at 5× SSC, 2% SDS, and 100 μg/ml single stranded DNA at 50° C. for 16 hours, followed by washing in 0.1× SSC, 0.1% SDS, at 50° C. for thirty minutes.

As used herein, the phrase “very high stringency conditions” refers the following conditions and equivalents thereto: hybridization at 5× SSC, 2% SDS, and 100 μg/ml single stranded DNA at 65° C. for 16 hours, followed by washing in 0.1× SSC, 0.1% SDS, at 65° C. for thirty minutes.

As used herein, the term “transcriptome” can be defined as the complete collection of transcribed elements of the genome. In addition to mRNAs, it also represents non-coding RNAs which are used for structural and regulatory purposes as well as splice variants, primary transcripts, and intermediates (hnRNA for example). Alterations in the structure or levels of expression of any one of these RNAs or their proteins can contribute to disease.

Heterologous Nucleic Acids of Interest

Sources

The present invention may be applied to rapid identification of oligonucleotides that hybridize to heterologous nucleic acids from a range of species. One of skill in the art would recognize the scope of organisms that the invention will apply to based upon the availability of commercial microarrays and of sequence data of homologues and orthologues where custom microarrays are used. One of skill in the art would further recognize that the breadth of organisms included in the definition heterologous will in part depend upon the nucleic acid of interest. It is well known that non-functional nucleic acid sequences will be more divergent than functional nucleic acid sequences and that even within functional nucleic acid sequences, the divergence varies. For example, heat shock genes show a higher degree of conservation across all organisms than many other genes owing to the essential nature of their function. Further, key regions within genes coding for functional regions of a protein will be more highly conserved than regions that code for structural regions of a protein. Thus, by careful selection or production of microarrays and the oligonucleotides on such arrays, one of skill in the art may tune the range of organisms that may be used in the present invention. Preferred heterologous organisms include cow, pig, sheep, horse, cat, dog, chicken, turkey, trout salmon, tilapia, catfish, shrimp, Drosophila, mosquitoes, wheat, corn, rice, barley, oat, rye, soybean, canola, cotton and their wild relatives. It also includes bacterial and viral disease agents affecting these organisms, as well as other organisms with unusual tolerance to environmental stressors, such as heat, drought, salinity or soil fertility.

In one embodiment of this invention, the nucleic acid of interest may be RNA or DNA, which is isolated from an organism of interest. In the case of RNA, the RNA can be isolated from an organ, tissue, or cell type of interest. In one version of this invention, RNA is an advantageous form of nucleic acid because RNA corresponds to expressed gene sequences, and hence gene transcripts, of the organism. The RNA can be converted to cDNA by methods well known in the art and as described herein in Example 2. Optionally, the cDNA can be made such that an anchor primer sequence is appended to the end of the resulting cDNA. Such an appended anchor sequence facilitates subsequent PCR cloning, as the anchor sequence can serve as one of two PCR primers. As will be appreciated by one of skill in the art, cDNA can be synthesized using oligo dT priming or by using random priming. As further described in Example 2, in one embodiment of this invention, the cDNA can be synthesized with an RNA polymerase promoter sequence such as T7, T3, or SP6 to allow for conversion of the cDNA sequence into cRNA for hybridization to a microarray. In addition, cDNA libraries are commercially available from a wide range of sources from a wide range of tissues.

When the nucleic acid of interest is an RNA molecule within the organism of interest's transcriptome, one of skill in the art may use any available expression data to aid in the selection of the appropriate organ, tissue, or cell-type as the source of the RNA. Preferred sources will be those sources where the RNA of interest has the highest relative expression as compared to other tissues, organs, cell-types, developmental stage, or environment. The environment would include any stimulus that may induce higher expression of the RNA of interest. By way of example, one of skill in the art would use tissue or cells exposed to heat stress when the RNA of interest codes for a heat shock protein. One of skill in the art may refer to existing expression profile data or may generate new expression profile data. The most preferred expression profile data is data generated from the microarray to be used in the later steps. Databases of existing expression profiles are available from numerous sources both commercial and non-commercial. Preferably, the expression data is generated by hybridizing expressed nucleic acids from the organism of interest to the chip with the heterologous sequences; however, expression data generated by hybridizing expressed nucleic acids from the heterologous organism to the chip with heterologous sequences will be useful as well. Alternatively, genomic DNA can be used in this invention. In this embodiment of the invention, total genomic DNA is isolated and fragmented. The fragmented DNA can be labeled and used to hybridize to a DNA microarray. An anchor sequence can be appended to molecules in a separate portion of the fragmented genomic DNA, to serve as a PCR primer for later PCR amplification.

The nucleic acid of this invention can be labeled using methods known in the art. Labels that can be used in this invention include fluorescence, radioactive, and biotin, to provide non-limiting examples. Examples of labeling methods include end labeling and incorporation of labeled nucleotides during nucleic acid synthesis.

Microarrays

After labeling, the nucleic acids are hybridized to microarrays of oligonucleotides derived from expressed transcript sequences of a species different from the species of interest under conditions that are known in the art. An example of such a condition is provided in Example 1. In various embodiments, useful microarrays can contain oligonucleotides derived from expressed transcript sequences of various organisms including without limitation human, mouse, rat, Arabidopsis, Drosophila, and C. elegans. Each expressed gene sequence can be represented on the microarray by one or more oligonucleotide probes per expressed transcript sequence. The preferred oligonucleotides have a length of between eight to sixty nucleotides. As a non-limiting example, the Affymetrix Human Genome U133A GeneChip as disclosed in Example 2 contained 11 sequences of human troponin C in which the oligonucleotides were 25 nucleotides in length.

Microarrays that can be generally useful in the practice of this invention can include microarrays that contain collections of oligonucleotides derived from the full complement of expressed genes in an organism, cell type, or tissue. The microarrays can contain single or multiple sequences derived from the sequences of particular or all expressed genes. Alternatively, the microarrays can contain a subset of the full complement of expressed genes. The oligonucleotides on the microarray can be of various lengths. In one embodiment of the invention, 16 bases is a minimal length of oligonucleotide used. Many microarrays suitable for use in the present invention are available commercially or can otherwise be fabricated according to methods well known in the art. Examples of commercially available microarrays that can be used in the practice of this invention include the Human Genome U133A GeneChip® and Mouse Genome 430A GeneChip® microarrays from Affymetrix (Affymetrix, Santa Clara, Calif.). Microarrays containing expressed genes from other organisms such as C. elegans and Drosophila are also commercially available and can be used in the practice of this invention.

As an alternative to commercially available microarrays, custom microarrays containing all or a subset of expressed gene sequences can be used in an embodiment of this invention. Such subsets of expressed gene sequences can include genes related to growth and development, diseases, disease resistance, cancer, and any other subsets of interest. These subsets can also be derived from the sequences of different organisms. The design of such custom arrays will depend upon the desired use of the array. For example, when cloning one or a limited set of nucleic acids, more oligonucleotides per nucleic acid may be designed for the array. More oligonucleotides will increase the likelihood of identifying an array oligonucleotide that will hybridize to the nucleic acid of interest. When the array is to be used to screen for a particular nucleic acid among a family of homologues, one of skill in the art would select sequences that are more unique to the nucleic acid of interest. When the array is to be used to screen for all nucleic acids within a family of homologues, one of skill in the art would select sequences that are more conserved within the family. In addition, a given array may contain sequences from one heterologous organism or from multiple. Multiple heterologous organisms may be preferred where the array is to be used to identify nucleic acids of interest from several organisms of interest. Finally, to increase the likelihood of success, multiple oligonucleotides that are degenerate versions of an oligonucleotide sequence in a heterologous organism may be used on the array to increase the likelihood of identifying an oligonucleotide that hybridizes to the nucleic acid of interest. One of skill in the art is familiar with and would apply all techniques in design of primers used in cloning related sequences in design of oligonucleotides for the custom array.

While the above discussion was directed to microarrays, one of skill in the art would recognize that the present invention is directed to the advantages of larger scale screening of which microarrays are only one example. The present invention may also be used with other related techniques such as micro-beads. Further, one of skill in the art would recognize that microarrays include all relevant forms of arrays including, without limitation, planar arrays, pin arrays, etc.

Detection of hybridization of the nucleic acid to an oligonucleotide of the array can be accomplished using various methods and systems known in the art.

Identification of Oligonucleotides

Following detection of hybridization, array oligonucleotides which have hybridized to a level suitable to act as probes are identified and selected using methods known in the art, such as by employing an electronic mask. As described in Example 1, an electronic mask can be used to eliminate poorly hybridized probes, i.e., ones that have a low difference between probes of perfect match versus mismatch or low ratio between probes of perfect match versus mismatch. In a preferred embodiment, the signal of the nucleic acid of interest hybridized to the array oligonucleotide is at least five times the standard deviation of the background noise.

Preferred Uses of the Oligonucleotides

The array oligonucleotides that hybridized to suitable levels have a wide range of uses. Oligonucleotides of the same sequence may, for example, be used (i) as probes for detection using any method available to one of skill in the art, (ii) as primers for sequencing, (iii) for purification by coupling the oligonucleotide to a solid support such as magnetic beads, and (iv) in a preferred embodiment, as primers for amplification of the nucleic acid of interest.

The identity of array oligonucleotides which have hybridized to suitable levels can be determined because the identity of the oligonucleotide at each position on the microarray is known. Oligonucleotides which correspond to genes of interest can then be selected as probes for further use.

PCR Amplification

Probes that show sufficient levels of hybridization to act as PCR primers can be used as one of a pair of PCR primers to amplify the corresponding expressed gene sequence from the species of interest. In an embodiment of the invention, the second PCR primer can correspond to anchor sequence appended to the end of the nucleic acid, with the nucleic acid having the appended anchor serving as the template. In a particular embodiment of the invention, the nucleic acid template is cDNA which has been synthesized with an appended anchor sequence during oligo-dT or random primed synthesis of first strand cDNA. In another embodiment, when at least two probes are identified, the antisense sequence of the probe that hybridizes to the nucleic acid of interest 3′ to the other probe may be used as the second primer.

The resultant PCR products can then be sequenced using methods known in the art. Typically, the PCR products will be cloned into suitable plasmid vectors for sequencing. Such cloning vectors are well known in the art.

In one embodiment, the optimal conditions for PCR may be screened by testing different hybridization conditions to the microarray. By screening different hybridization conditions to the microarray, conditions can be identified which minimize non-specific hybridization while maximizing the specific hybridization between the nucleic acid of interest and array oligonucleotides. One of skill in the art is aware of many parameters that may be manipulated to alter the hybridization in addition to altering the sequence of the array oligonucleotide. Examples of such parameters include temperature, salt concentration, divalent cation concentrations, pH, and various additives such as dimethyl sulfoxide, non-ionic detergents (Triton X-100, Tween-20, etc.), and Betaine (to reduce or eliminate the difference in contribution to stability of the nucleic acid duplex between G-C and A-T base pair). Such optimized conditions may be used in PCR to enhance amplification of the nucleic acid of interest. In addition, such optimization may be used in conjunction with other uses of the oligonucleotides.

In another embodiment, the PCR may be performed directly on the array. The standard techniques of PCR may be adapted to target nucleic acids hybridized to microarrays allowing amplification of entire libraries of expressed nucleic acids from the organism of interest. Recent examples of on-chip PCR have demonstrated the efficacy of the technique. (see Anal. Biochem. 303:25 (2002) and Biotechniques 29:844 (2000))

In yet another embodiment, the expressed nucleic acids from the organism of interest may be sequenced directly on the microarray. One of skill in the art could adapt existing methods and equipment to allow for on-chip sequencing. By way of example, one of skill in the art could sequence expressed nucleic acids of interest by (1) hybridizing the expressed nucleic acids of interest to the microarray, (2) adding polymerase with dNTPs that are each labeled with a different fluorophore such as the Rhodamine label system used in automated sequencing (ideally, the fluorophore would be a removable label that blocks addition of further nucleotides), (3) scanning the microarray to determine which base was added for each expressed nucleotide of interest, (4) removing the blocking label and repeating from step (2) sequentially to sequence the expressed nucleotides of interest. The advantage of such on-chip sequencing would be that potentially thousands of expressed nucleotides could be expressed simultaneously.

Hybridization Probes

In another embodiment of the invention, the oligonucleotide(s) of the microarray which are identified from hybridization of the nucleic acid from a species of interest to the microarray can be used as probes to directly screen a cDNA or genomic library without PCR amplification. Methods for screening cDNA or genomic libraries with oligonucleotides are well known in the art.

According to a preferred embodiment of the invention, total RNA of a species under investigation is isolated. Complementary DNA (cDNA) is synthesized by reverse transcriptase. T7 RNA polymerase promoter and (or) anchoring PCR primer are incorporated into 5′ end of the cDNA via connecting with an oligo d(T) primer. Complementary RNA (cRNA) is synthesized by in vitro transcription with T7 RNA polymerase. The cRNA is fragmented before hybridized with an oligonucleotide microarray containing oligonucleotides of sequences corresponding to all or a subset of expressed gene sequences in the genome of a particular species. Using methods known in the art, the microarray images are processed and hybridization signals of each microarray probe are calculated. An electronic mask is created to remove poorly hybridized probes that have either low PM-MM (difference between probes of perfect match and mismatch) or low PM/MM (ratio between probes of perfect match and mismatch). The probes that have passed the electronic mask will be selected and used directly for PCR amplification of heterologous genes. The PCR amplification uses two primers, an anchoring primer that has been incorporated during cDNA synthesis, and a target specific primer that is directly obtained from the GeneChip probe. After the PCR amplification, the heterologous genes are sequenced by dideoxynucleotide termination methods.

The present invention overcomes the problems and difficulties associated with large scale DNA sequencing. In the Examples described, cRNAs from distant mammals, such as cattle, dog, pig, were able to hybridize with oligonucleotide microarrays of genes transcribed from the human genome quite specifically. Generally, a perfect match of 16 base-pair is required for the heterologous hybridization to generate measurable signals on an Affymetrix gene Chip. Thus, the heterologous hybridization can be used to interrogate the sequences of a species under investigation in a large scale and parallel way. Heterologous hybridization allows for the identification of the sections where the heterologous gene is identical to its human ortholog, whose sequence is already available. Heterologous hybridization also allows for target-specific amplification of a heterologous gene by PCR with two primers, an anchoring primer that was incorporated into every transcript during cDNA synthesis and a primer derived directly from the oligonucleotide probe whereupon heterologous hybridization took place. Further, the amount of PCR product generated positively correlates with the signal strength of the heterologous hybridization. Therefore, the information of the heterologous hybridization can be utilized extensively for the gene amplification of an investigative species.

### PREFERRED USES OF THE PRESENT INVENTION

The present invention has several important features and advantages, including but not limited to: 1) providing a comprehensive coverage of the transcriptome of the species of interest, given that heterologous hybridization between distant mammals has been observed to take place on thousands of different transcripts; 2) sequencing targets are completely normalized, because typically all genes are represented equally on microarrays of expressed genes from the genome of an organism; 3) the sequencing targets are annotated beforehand, because their human orthologs are placed in precise and known locations on the microarray; 4) designing PCR primers is no longer necessary, because they are derived directly from the known microarray probes.

The methods of the present invention may be used for a wide range of purposes. The methods may be applied to a single organism of interest to identify heterologous oligonucleotides that hybridize to a single nucleotide of interest. However, in preferred embodiments, the methods are used to identify at least two, at least three, at least four, at least five, at least ten, at least twenty, at least fifty, at least one hundred, or at least five hundred oligonucleotides, where each oligonucleotide hybridizes to a different nucleic acid of interest. Thus, the present invention may be applied to cloning and/or sequencing particular genes of interest. In addition, the present invention may be applied to large scale sequencing of at least a significant portion of an organism's transcriptome which may in turn be used to create new microarrays for the specific organism of interest for expression profiling, etc.

In addition, the methods may be applied to multiple organisms by use of multiple microarrays. Cloning and/or sequencing a gene of interest from multiple related organisms may be beneficial for a variety of reasons. When studying a gene of interest, sequence alignments of related genes can provide a wealth of information as to function given that functional regions will show greater degrees of conservation. Also, where the gene of interest is an enzyme for example, multiple homologues may be screened for optimal catalytic parameters, substrate specificity, protein stability, etc. Finally, in cases where no sequence data exists from another organism that is close enough to the organism of interest for the present invention to succeed, the present invention may be applied to an intermediate organism that is related to both the organism of interest and to an organism where the nucleic acid of interest has been sequenced. With sequence data of the nucleic acid of interest from the intermediate organism in hand, a custom array may be designed to identify oligonucleotides that hybridize to the nucleic acid of interest from the organism of interest.

The following examples of the present invention are merely exemplary in nature and, thus, one of skill in the art would understand that the present includes all variations that do not depart from the basic scope of the invention as set forth herein. Such variations are intended to be within the scope of the present invention and are therefore not to be regarded as a departure from the spirit and scope of the invention.

### EXAMPLE 1

### Performing Heterologous GeneChip Hybridization

### Microarrays and RNA

Human Genome U133A GeneChip® and Mouse Genome 430A GeneChip® microarrays were purchased from Affymetrix (Affymetrix, Santa Clara, Calif.). Human heart and liver total RNAs were purchased from Clontech (Clontech, Palo Alto, Calif., USA). Heart and liver of cattle, dog and pig were obtained from freshly slaughtered carcasses. Total RNAs were isolated from heart and liver tissues by a method described Gauthier et al (see Pflugers Arch. 433: 664 (1997)). One hundred micrograms of total RNA were treated with 1.0 Unit of DNAse I (Amplification grade, Gibco-BRL, Bethesda, Md., USA) at 37° C. for 15 min. The RNAs were further purified using RNeasy Mini columns (Qiagen, Chatsworth, Calif., USA).

Preparation of cDNA

Complementary DNA (cDNA) was prepared with cDNA Synthesis System Kit purchased from Roche Diagnostics (Roche Diagnostics GmbH, Mannheim, Germany). In short, a mixture, containing 20 μg of total RNA, 200 μmol of oligo{(dT)24T7promotor}65 primer, and ddH2O in a volume of 21 μl, was incubated at 70° C. for 10 min, then placed on ice. The first-strand cDNA was synthesized by adding following reagents to the mixture, 8 μl of 5× RT buffer, 4 μl of 0.1 M DTT, 4 μl of 10 mM dNTP, 1 μl of RNase inhibitor (25 U/μl), and 2 μl of AMV reverse transcriptase (25 U/μl). The reaction was incubated at 42° C. for 60 min and terminated by cooling on ice.

The second strand cDNA was synthesized with a reaction mixture containing 40 μl of the first strand cDNA reaction, 72 μl of ddH2O, 30 μl of 5× 2nd strand buffer, 1.5 μl of 10 mM dNTP, 6.5 μl of 2nd strand enzyme blend consisting of DNA polymerase I (80 U), E. coli ligase (20 U), and RNase H(4 U). The reaction was incubated at 16° C. for 2 hr. The reaction was stopped by adding 17 μl of 0.2M EDTA (pH8.0).

The dscDNA preparation was digested with 1.5 μl of RNase I (10 U/μl) at 37° C. for 30 min to remove residual RNA, and subsequently treated with 5 μl of Proteinase K (0.6 U/μl) at 37° C. for 30 min.

The dscDNA preparation was extracted sequentially with 200 μl of phenol, 200 μl of phenol/chloroform/isoamyl alcohol (25/24/1), and twice with 200 μl of chloroform/isoamyl alcohol (24:1). The supernatant was saved and mixed with 0.6 vol. of 5 M NH4OAc, and then with 2.5 vol. of chilled alcohol. It was kept at −60° C. for 1 hr to precipitate dscDNA. The mixture was centrifuged at 10,000 g for 10 min. The pellet was washed with 300 μl of 80% alcohol, and then air-dried. The dscDNA was dissolved in 1.5 μl of ddH2O.

Preparation of Biotin-Labeled cRNA

Biotin-labeled nucleotide Bio-11-CTP and Bio-16-UTP were purchased from Enzo Biochem (Enzo Biochem, New York, N.Y.). T7 RNA polymerase MEGAscript T7 Kit was purchased from Ambion (Ambion, Austin, Tex.). Reaction mixture contained 2.0 μl of 10× T7 RNA polymerase buffer, 2.0 μl of 75 mM ATP, 2.0 μl of 75 mM GTP, 1.5 μl of 75 mM CTP, 1.5 μl of 75 mM UTP, 3.75 μl of 10 mM Bio-1-CTP, 3.75 μl of 10 mM Bio-16-UTP, 2.0 μl of 10× T7 RNA polymerase enzyme mix, and 1.5 μl of cDNA (as prepared above). The reaction mixture was incubated at 37° C. for 5 hr. The labeled cRNA was purified with RNeasy Mini kit and eluted in 50 μl of ddH2O. It was fragmented at 95° C. for 35 min in a solution containing 40 mM Tris-acetate (pH 8.1), 100 mM KOAc, and 30 mM MgOAc. The fragmented cRNA was used either immediately for chip hybridization or stored in a −80° C. freezer.

Hybridization with Affymetrix GeneChips®

A hybridization mix consisting of 50 μg of fragmented cRNA, 125 μl of 2× MES buffer (0.2 M MES pH6.7, 2M NaCl, 0.02% Triton), 6.25 μl of acetylated BSA (20 μg/μl), 2.5 μl of herring sperm DNA (10 μg/μl), 2.5 μl of biotinylated Control Oligo (5 nM, Affymetrix, Santa Clara, Calif.), and ddH2O in a total volume of 250 μl, was heated at 95° C. for 5 min and then allowed to equilibrate at 45 C

Microarray chips were first treated at 45° C. for 5 min with 250 μl of prehybridization solution consisting of 125 μl of 2× MES buffer and 125 μl of ddH2O. They were then incubated with the hybridization solution at 45° C. for 16 hr in a rotary agitation hybridization oven.

### GeneChip Washing, Staining and Scanning

After the removal of hybridization solution, the chips were washed with 6× SSPE-T (0.9M NaCl, 0.06M NaH2PO4 pH 6.7, 6 mM EDTA and 0.01% Triton) for ten times using the Affymetrix Fluidics Station. Chips were rinsed once with 0.1× MES and then incubated with 0.1× MES at. 45° C. for 15 min in a rotary oven. The chips were rinsed with 1× MES before being stained with 220 μl of staining solution containing 205 μl of 1× MES, 23 μl of acetylated BSA (20 μg/μl), and 2.3 μl of phycoerythrin-strepavidin conjugate (1 mg/ml) (Molecular Probes, Eugene, Oreg.). Chips were incubated in staining solution at 35° C. for 15 min in a rotary oven. The chips were subsequently washed with 6× SSPE-T ten times on an Affymetrix Fluidic Station. The chips were immediately scanned on a HP GeneArray Scanner.

### GeneChip Image Quantification and Data Processing

GeneChip images were quantified and gene expression values were calculated by Affymetrix Microarray Suite Version 5.0 (MAS 5.0). Individual electronic masks were generated by Affymetrix MAS 5.0.

The heterologous hybridization results are demonstrated in following figures.

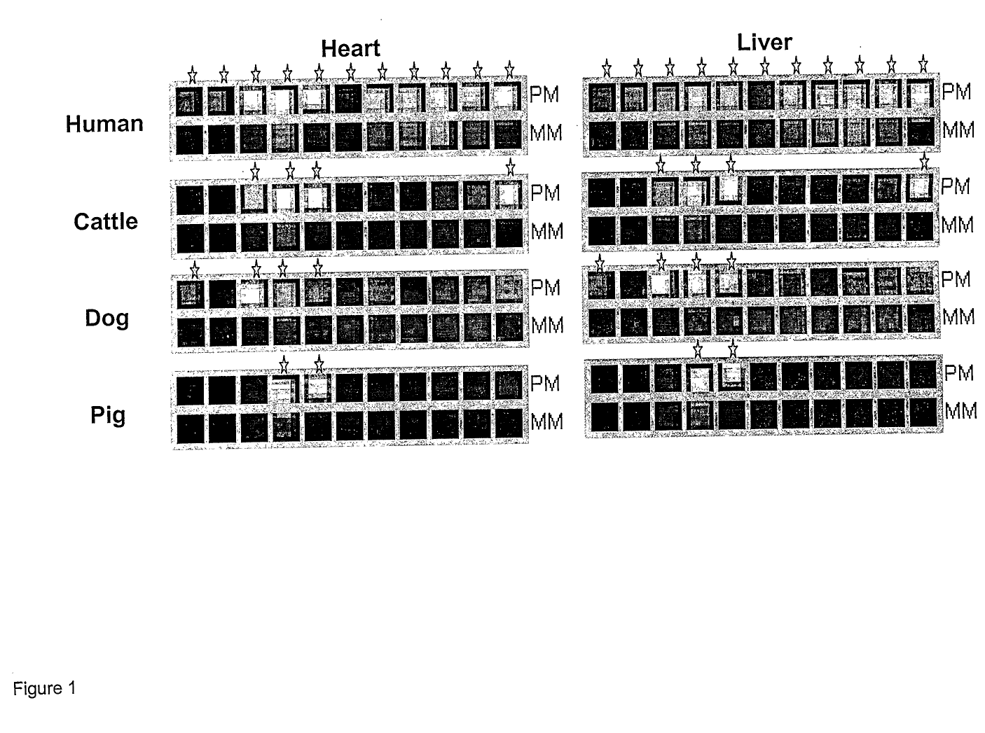

FIG. 1. shows GeneChip Human Genome U133A hybridization images of ribosomal protein L37a from heart and liver of human, cattle, dog, and pig. PM and MM denote perfect match probes and mismatch probes respectively. The probes that produce a relatively high hybridization signal are labeled with a star.

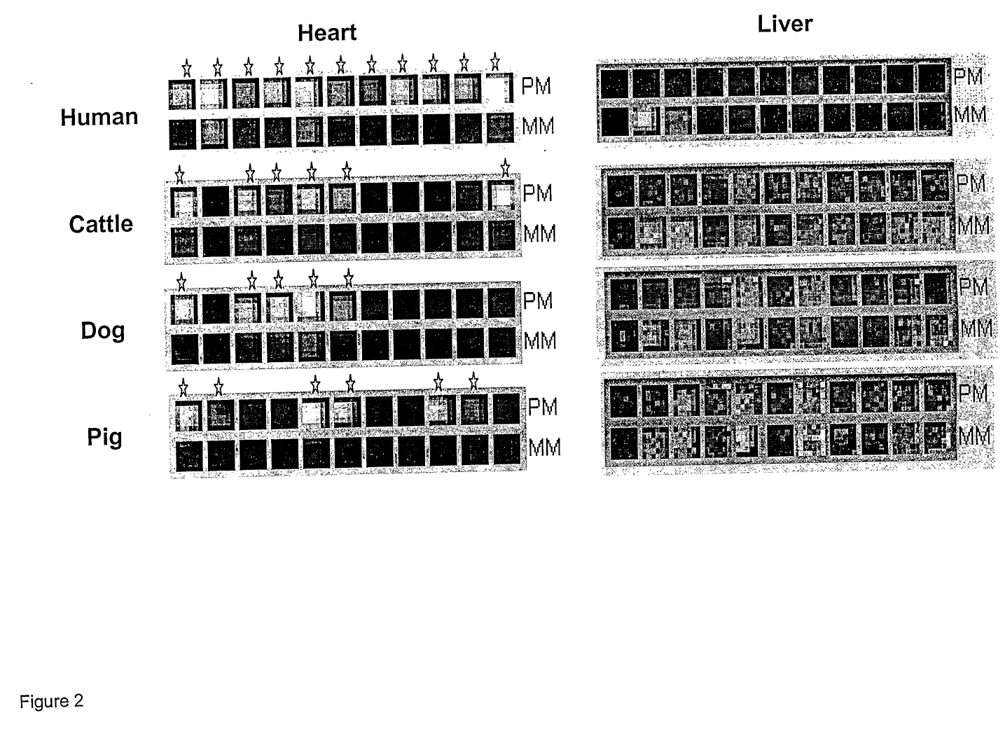

FIG. 2. shows GeneChip Human Genome U133A hybridization images of troponin C from heart and liver of human, cattle, dog, and pig. PM and MM denote perfect match probes and mismatch probes respectively. The probes that produce a relatively high hybridization signal are labeled with a star.

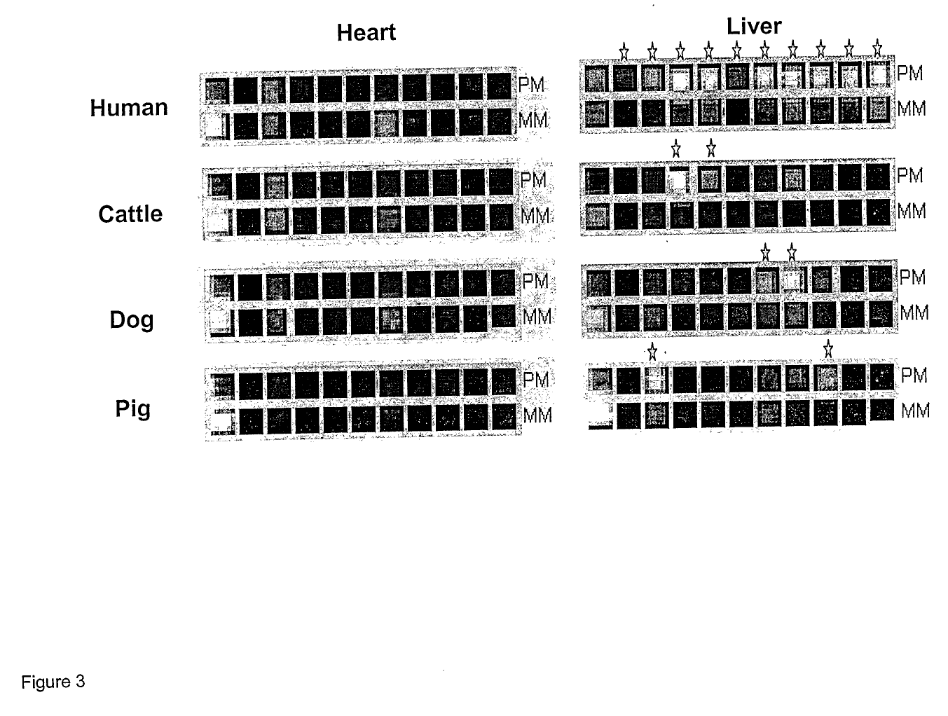

FIG. 3. shows GeneChip Human Genome U133A hybridization images of apolipoprotein C-III from heart and liver of human, cattle, dog, and pig. PM and MM denote perfect match probes and mismatch probes respectively. The probes that produce a relatively high hybridization signal are labeled with a star.

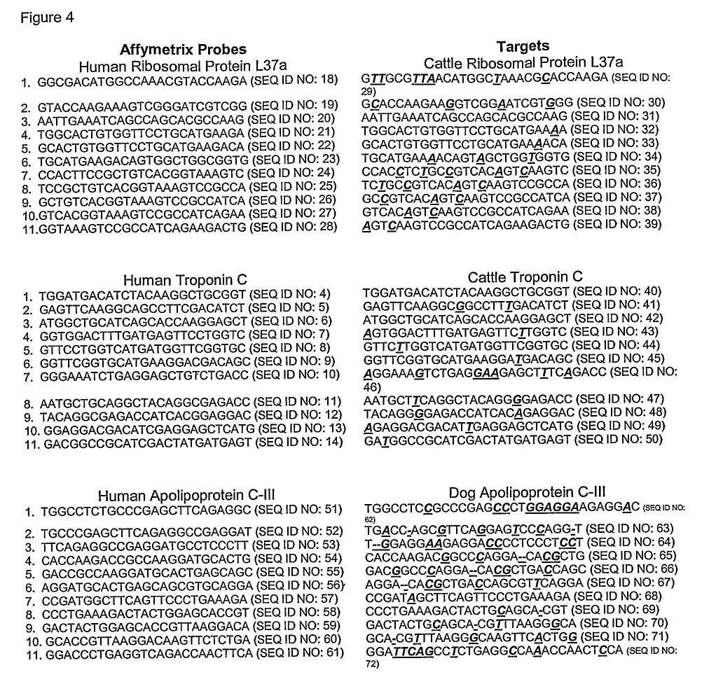

FIG. 4. is a sequence comparison of GeneChip probes and their heterologous targets. The mutated nucleotides are labeled with bold, italic and underlined fonts. The probes that produce a relatively high hybridization signal are labeled with a star.

The heterologous GeneChip hybridization demonstrated that cRNAs from distant mammals, such as cattle, dog, pig, were able to hybridize with Affymetrix human GeneChips quite specifically. Generally, a perfect match of 16 base-pair is required for the heterologous hybridization to generate measurable signals. Thus, the heterologous GeneChip hybridization can be used to interrogate the sequences of an investigative species in a massive parallel way. Albeit its incompleteness, we could at least be able to identify the sections where the heterologous gene is identical to its human ortholog whose sequence is already available.

### EXAMPLE 2

### PCR Amplification of Target Genes with the Primers Derived from Affymetrix GeneChip Probes

In this example, we performed both GeneChip hybridization and targeted gene amplification from as little as 100 ng of total RNA.

### Microarray, Oligonucleotides and RNA

Human Genome U133A GeneChip® microarrays were purchased from Affymetrix (Affymetrix, Santa Clara, Calif.). Oligonucleotides were all purchased from IDT (IDT, Coraville, Iowa). Their sequences are: PCRT7(T)24: AAGCAGTGGTAACAACGCAGAGTGAATTAATACGACTCACTATAGGGAGA(T) 24VN (SEQ ID NO: 1); Smart II: AAGCAGTGGTAACAACGCAGAGTACGCGGG (SEQ ID NO: 2); PCR primer: AAGCAGTGGTAACAACGCAGAGT (SEQ ID NO: 3). Human heart total RNAs was purchased from Clontech (Clontech, Palo Alto, Calif., USA). Heart of cattle and dog were obtained from freshly slaughtered carcasses. Total RNAs were isolated from heart and liver tissues by a method described Gauthier et al (see Pflugers Arch. 433: 664 (1997)). One hundred micrograms of total RNA were treated with 1.0 Unit of DNAse I (Amplification grade, Gibco-BRL, Bethesda, Md., USA) at 37° C. for 15 min. The RNAs were further purified using RNeasy Mini columns (Qiagen, Chatsworth, Calif., USA).

### Preparation of PCRcDNA

First-strand cDNA was prepared by a modification of the methods described in the SMART PCR cDNA synthesis kits (Clontech, Palo Alto, Calif., USA). A mixture, consisting of 100 ng of total RNA, 1.0 μl of 20 μM PCRT7(T)24 primer, 1.0 μl of 20 μM SMART II primer, and ddH2O in a volume of 9.0 μl, was heated at 70° C. for 2 min and then kept at 0° C. A mixture, containing 4.0 μl of 5× reverse transcriptase buffer, 1.0 μl of 0.2 M DTT, 2.0 μl of 10 mM dNTP, 1.0 μl of RNase OUT (40 unit), and 1.0 μl of Superscript II reverse transcriptase (200 unit) (Life Technology, Rockville, Md., U.S.A.), was added. The reverse transcriptase reaction was incubated at 42° C. for 1 hr. It was terminated by 80 μl of TE buffer followed by incubation at 72° C. for 7 min. It was then mixed with 1.0 μl of glycogen (20 μg/μl, Sigma, St. Louis, Mo.), 10.0 μl of 5 M NH4OAc, and 250 μl of ethanol. The mixture was incubated at −20° C. for 15 min and then centrifuged at 10,000× g for 10 min. The pellet was washed with 70% ethanol, air dried and dissolved in 10 μl of ddH2O.

The PCR reaction mixture contained 10.0 μl of first-strand cDNA (prepared as above), 5.0 μl of 10× PCR reaction buffer, 1.0 μl of 10 mM dNTPs, 2.0 μl of 10 μM PCR primer, and 1.0 μl of Klentaq Advantage Taq DNA polymerase in a volume of 50.0 μl (Clontech, Palo Alto, Calif.). The PCR amplification was performed with the following cycling parameters: 95° C. 1 min, then 26 cycles of 95° C. 15 sec, 65° C. 30 sec and 68° C. 3 min, in a MJ Research Tetrad thermocycler (MJ Research, Waltham, Mass.).

The PCRcDNA preparation was purified with QIAquick PCR Purification columns (Qiagen, Chatsworth, Calif., USA) and eluted in 50.0 μl of ddH2O. It was further purified by ethanol precipitation by adding to the elutant 1.0 μl of glycogen, 5.0 μl of 5 M NH4OAc and 150 μl of ethanol. The mixture was incubated at −20° C. for 15 min and then centrifuged at 10,000×g for 10 min. The pellet was washed with 70% ethanol, air dried and dissolved in 7.5 μl of ddH2O.

### Preparation of Biotin-Labeled PCRcRNA

Biotin-labeled nucleotide Bio-11-CTP and Bio-16-UTP were purchased from Enzo Biochem (Enzo Biochem, New York, N.Y.). The T7 RNA polymerase MEGAscript T7 Kit was purchased from Ambion (Ambion, Austin, Tex.). Reaction mixtures contained 2.0 μl of 10× T7 RNA polymerase buffer, 2.0 μl of 75 mM ATP, 2.0 μl of 75 mM GTP, 1.5 μl of 75 mM CTP, 1.5 μl of 75 mM UTP, 3.75 μl of 10 mM Bio-11-CTP, 3.75 μl of 10 mM Bio-16-UTP, 2.0 μl of 10× T7 RNA polymerase enzyme mix, and 1.5 μl of PCRcDNA (as prepared above). The reaction mixture was incubated at 37° C. for 5 hr. The labeled cRNA was purified with an RNeasy Mini kit and eluted in 50 μl of ddH2O. It was fragmented at 95° C. for 35 min in a solution containing 40 mM Tris-acetate (pH 8.1), 100 mM KOAc, and 30 mM MgOAc. The fragmented cRNA was used either immediately for chip hybridization or stored in a −80° C. freezer.

### Hybridization Against Affymetrix GeneChips®

A hybridization mixture, consisting of 50 μg of fragmented cRNA, 125 μl of 2× MES buffer (0.2 M MES pH6.7, 2M NaCl, 0.02% Triton), 6.25 μl of acetylated BSA (20 μg/μl), 2.5 μl of herring sperm DNA (10 μg/μl), 2.5 μl of biotinylated Control Oligo (5 nM,), and ddH2O in a total volume of 250 μl, was heated at 95° C. for 5 min and then allowed to equilibrate at 45° C. Affymetrix HG U133A Gene Chips were first treated at 45° C. for 5 min with 250 μl of prehybridization solution consisting of 125 μl of 2× MES buffer and 123 μl of ddH2O. They were then incubated with the hybridization solution at 45° C. for 16 hr in a rotary agitation hybridization oven.

### GeneChip Washing, Staining and Scanning

After the hybridization and removal of hybridization solution, the chips were washed with 6× SSPE-T (0.9M NaCl, 0.06M NaH2PO4 pH 6.7, 6 mM EDTA and 0.01% Triton) for ten times using the Affymetrix Fluidics Station. Chips were rinsed once with 0.1× MES and then incubated with 0.1× MES at 45° C. for 15 min in a rotary oven. The chips were rinsed with 1× MES before being stained with 220 μl of staining solution containing 205 μl of 1× MES, 23 μl of acetylated BSA (20 μg/μl), and 2.3 μl of phycoerythrin-strepavidin conjugate (1 mg/ml) (Molecular Probes, Eugene, Oreg.). Chips were incubated in staining solution at 35° C. for 15 min in a rotary oven. The chips were subsequently washed with 6× SSPE-T for ten times on Affymetrix Fluidic Station. Chips were immediately scanned on a HP GeneArray Scanner.

### GeneChip Image Quantification and Data Processing

GeneChip images were quantified and calculated by Affymetrix Microarray Suite Version 5.0.

Amplification of Troponin C from Human, Cattle and Dog

PCRcDNA of human, cattle and dog as described above were diluted 1000 fold and 10 μl of the dilute product was used for 2nd PCR amplification. The 2nd PCR reaction mixture contained 10.0 μl of 1st PCR dilute, 5.0 μl of 10×PCR reaction buffer, 1.0 μl of 10 mM dNTPs, 2.0 μl of 10 μM PCR primer, and 1.0 μl of Klentaq Advantage Taq DNA polymerase in a volume of 50.0 μl (Clontech, Palo Alto, Calif.). The PCR amplification was performed with the following cycling parameters: 95° C. 1 min, then 15 cycles of 95° C. 15 sec and 68° C. 30 sec, in a MJ Research Tetrad thermocycler (MJ Research, Waltham, Mass.). The 2nd PCR products were diluted 100 fold and the dilute product was used for PCR amplification of target genes.

The sequences of the 11 probes of human troponin C on Human Genome U133A GeneChip were obtained from Affymetrix website (available online at affymetrix.com).

| | | |
|---|---|---|
| They are: | | |
| TropoC1 | 5′TGGATGACATCTACAAGGCTGCGGT3′, | (SEQ ID NO: 4) |
| TropoC2 | 5′GAGTTCAAGGCAGCCTTCGACATCT3′, | (SEQ ID NO: 5) |
| TropoC3 | 5′ATGGCTGCATCAGCACCAAGGAGCT3′, | (SEQ ID NO: 6) |
| TropoC4 | 5′GGTGGACTTTGATGAGTTCCTGGTC3′, | (SEQ ID NO: 7) |
| TropoC5 | 5′GTTCCTGGTCATGATGGTTCGGTGC3′, | (SEQ ID NO: 8) |
| TropoC6 | 5′GGTTCGGTGCATGAAGGACGACAGC3′, | (SEQ ID NO: 9) |
| TropoC7 | 5′GGGAAATCTGAGGAGCTGTCTGACC3′ | (SEQ ID NO: 10) |
| TropoC8 | 5′AATGCTGCAGGCTACAGGCGAGACC3′, | (SEQ ID NO: 11) |
| TropoC9 | 5′TACAGGCGAGACCATCACGGAGGAC3′, | (SEQ ID NO: 12) |
| TropoC10 | 5′GGAGGACGACATCGAGGAGCTCATG3′, | (SEQ ID NO: 13) |
| TropoC11 | 5′GACGGCCGCATCGACTATGATGAGT3′. | (SEQ ID NO: 14) |

The anchoring primer sequence is 5′CGCAGAGTGAATTAATACGACTCACT3′ (SEQ ID NO: 15). PCR reaction for target gene amplification contained 10.0 μl of 2nd PCR dilute, 5.0 μl of 10× PCR reaction buffer, 1.0 μl of 10 mM dNTPs, 1.5 μl of 10 μM anchoring primer, 1.5 μl of 10 μM Troponin C primer (from 1 to 10), and 1.0 μl of Klentaq Advantage Taq DNA polymerase in a volume of 50.0 μl (Clontech, Palo Alto, Calif.). The PCR amplification was performed with the following cycling parameters: 95° C. 1 min, then 23 cycles of 95° C. 15 sec and 68° C. 30 sec, in a MJ Research Tetrad thermocycler (MJ Research, Waltham, Mass.).

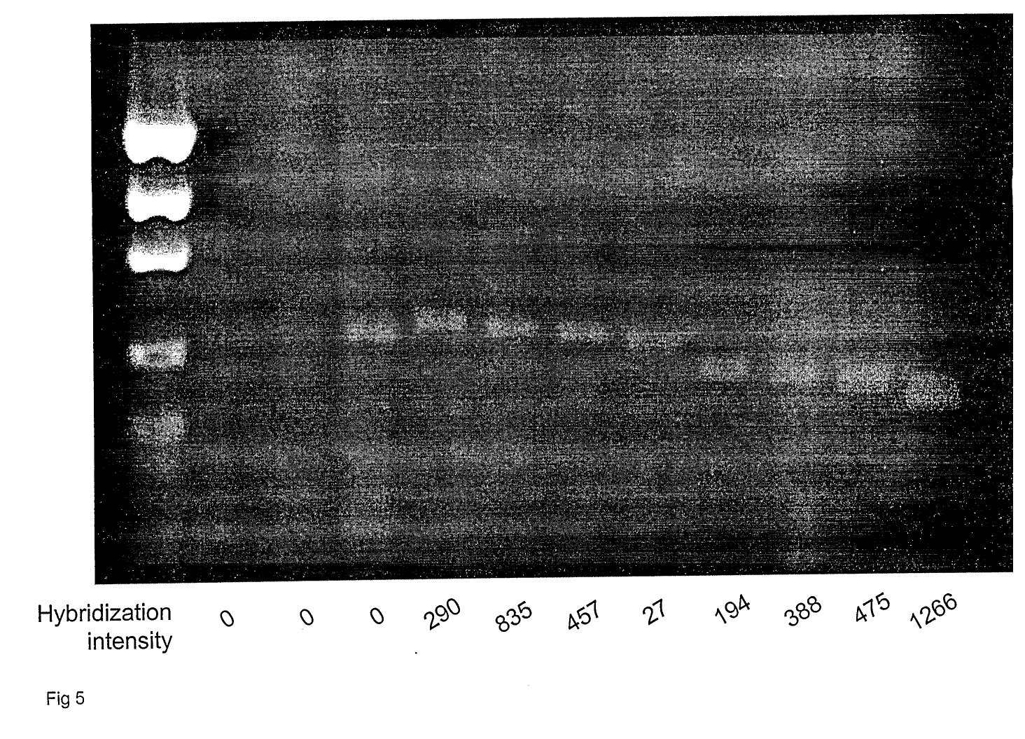

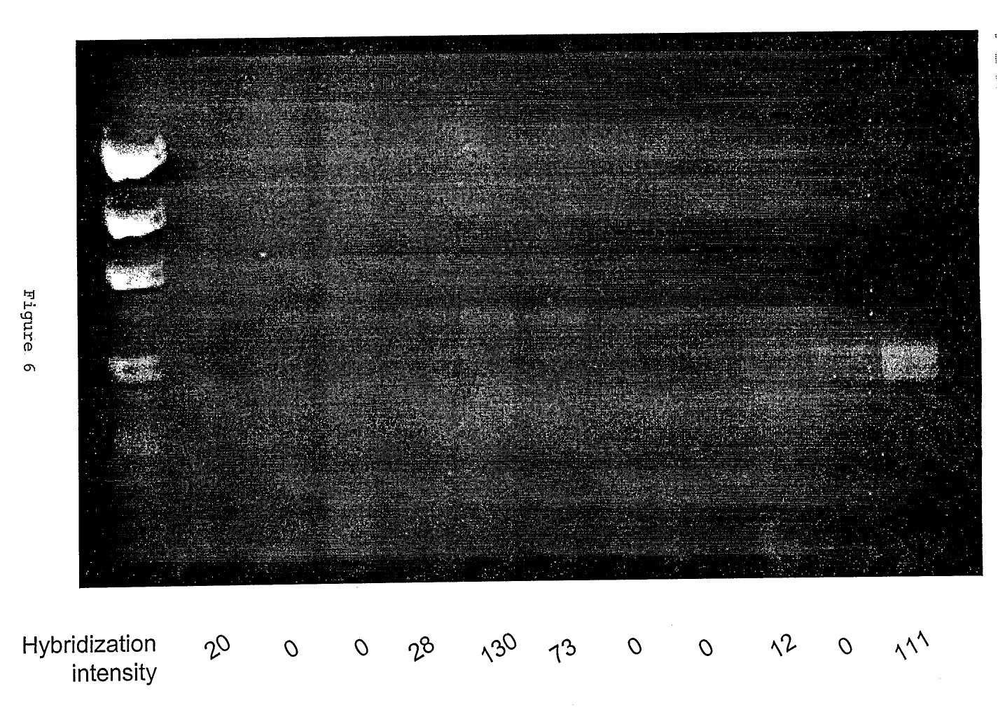

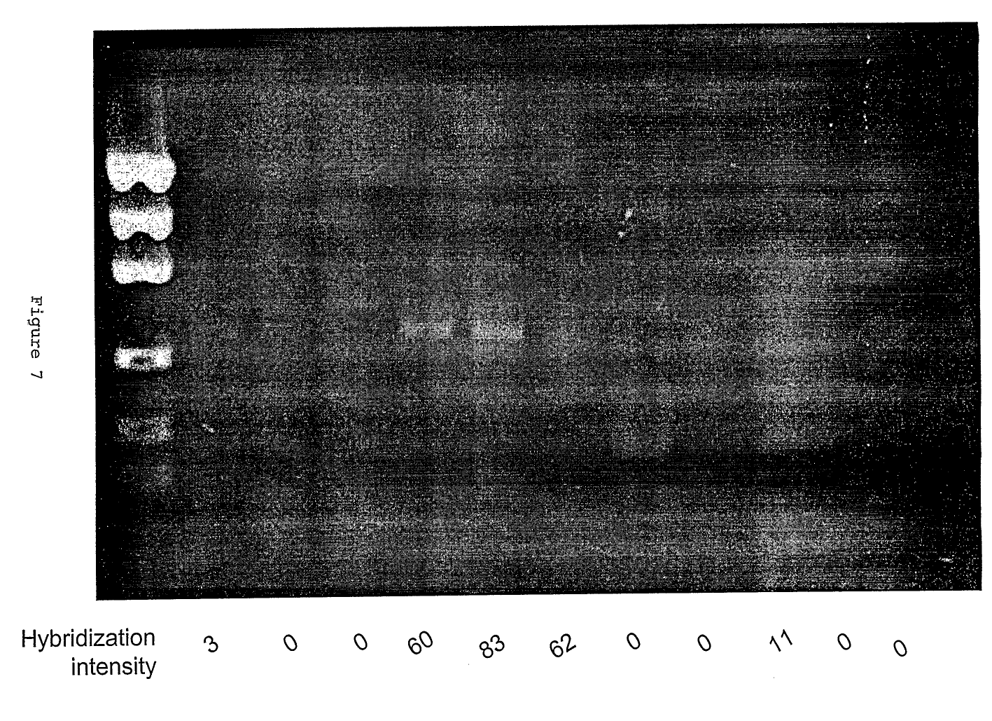

FIGS. 5, 6 and 7 demonstrate that the probes that generated high hybridization signals consistently produced more PCR products. Therefore, heterologous GeneChip hybridization can provide guidance for PCR amplification of the genes from an investigative species.

### EXAMPLE 3

### Custom DNA Microarrays

Custom DNA microarrays can also be generated for use in this invention as follows. Subsets of the genes known to be expressed in humans or other organisms of interest can be selected from databases of genes which have been sequenced. In particular, sequence information is available as a result of the complete or extensive sequencing of the human, mouse, rat, Drosophila, C. elegans, Arabidopsis, and zebrafish genomes, among others. Genes chosen for inclusion on a DNA microarray can correspond to any phenotype or characteristic of interest. Examples of such characteristics or phenotypes that can form subsets for a custom microarray include genes responsible for growth and development, disease causation, disease resistance, and cancer. Examples of genes involved in growth and development include: cyclins, E2Fs, cyclin dependent kinases, etc. Various numbers of oligonucleotides corresponding to distinct or overlapping sequences from each gene in the subset can be placed on the microarray. Preferably between 2 and 10 oligonucleotides per gene will be placed in defined locations on the microarray. Oligonucleotides corresponding to genes to be represented on the microarray can be synthesized using oligonucleotide synthesis methods well known in the art. The oligonucleotides preferably have a length of 8 to 60 bases. The oligonucleotides can be placed on the microarray using microarray fabrication and spotting methods well known in the art such as those described in U.S. Pat. Nos. 5,807,522 and 6,110,426.

### EXAMPLE 4

### Target Gene Sequencing with the Primers Derived from Affymetrix GeneChip Probes and Sequence Comparison Among Species

### Microarray, Oligonucleotides and RNA

Arabidopsis ATH1 genome arrays were purchased from Affymetrix (Affymetrix, Santa Clara, Calif.). Oligonucleotides were all purchased from IDT (IDT, Coraville, Iowa). Their sequences are:

PCRT7(T)24:

(SEQ ID NO: 1)

AAGCAGTGGTAACAACGCAGAGTGAATTAATACGACTCACTATAGGGAGA
(T) 24VN;
Smart II:

(SEQ ID NO: 2)

AAGCAGTGGTAACAACGCAGAGTACGCGGG;
PCR primer:

(SEQ ID NO: 3)

AAGCAGTGGTAACAACGCAGAGT.

Fresh leaves of Arabodopsis thaliana, Cauliflower, Napa and Brassica rapa were collected. Total RNAs were isolated from leaves with a modified method described by Gauthier et al (see Pflugers Arch. 433: 664 (1997)). The leaves were homogenized in 0.8 ml GTC solution (4 M guanidine thiocyanate, 25 mM sodium citrate, 0.5% N-lauroylsarcosine) supplemented with 0.7% β-mercaptoethanol. 80 μl of 2M sodium acetate (pH4) was added to the homogenate followed by addition of 0.8 ml of phenol (saturated with 0.1 M citrate buffer pH4.3), and 0.16 ml of chloroform:isoamyl alcohol (24:1). The mixture was placed on ice for 15 minutes, and then centrifuged at 10,000×g for 10 minutes. The resulting aqueous phase was transferred to a new tube, to which 1 volume of 70% ethanol was added. The ethanol mixture was then loaded to RNeasy Mini columns for a further purification (Qiagen, Chatsworth, Calif., USA).

### Preparation of PCRcDNA

First-strand cDNA was prepared by a modification of the methods described in the SMART PCR cDNA synthesis kits (Clontech, Palo Alto, Calif., USA). A mixture, consisting of 100 ng of total RNA, 1.0 μl of 20 μM PCRT7(T)24 primer, 1.0 μl of 20 μM SMART II primer, and ddH2O in a volume of 9.0 μl, was heated at 70° C. for 2 min and then kept at 0° C. A mixture, containing 4.0 μl of 5× reverse transcriptase buffer, 1.0 μl of 0.2 M DTT, 2.0 μl of 10 mM dNTP, 1.0 μl of RNase OUT (40 unit), and 1.0 μl of Superscript II reverse transcriptase (200 unit) (Life Technology, Rockville, Md., U.S.A.), was added. The reverse transcriptase reaction was incubated at 42° C. for 1 hr. It was terminated by 80 μl of TE buffer followed by incubation at 72° C. for 7 min. It was then mixed with 1.0 μl of glycogen (20 μg/μl, Sigma, St. Louis, Mo.), 10.0 μl of 5 M NH4OAc, and 250 μl of ethanol. The mixture was incubated at −20° C. for 15 min and then centrifuged at 10,000× g for 10 min. The pellet was washed with 70% ethanol, air dried and dissolved in 10 μl of ddH2O.

The PCR reaction mixture contained 10.0 μl of first-strand cDNA (prepared as above), 5.0 μl of 10× PCR reaction buffer, 1.0 μl of 10 mM dNTPs, 2.0 μl of 10 μM PCR primer, and 1.0 μl of Advantage Taq DNA polymerase in a volume of 50.0 μl (Clontech, Palo Alto, Calif.). The PCR amplification was performed with the following cycling parameters: 95° C. 1 min, then 26 cycles of 95° C. 15 sec, 65° C. 30 sec and 68° C. 3 min, in a MJ Research Tetrad thermocycler (MJ Research, Waltham, Mass.).

The PCRcDNA preparation was purified with QIAquick PCR Purification columns (Qiagen, Chatsworth, Calif.) and eluted in 50.0 μl of ddH2O. It was further purified by ethanol precipitation by adding to the elutant 1.0 μl of glycogen, 5.0 μl of 5 M NH₄OAc and 150 μl of ethanol. The mixture was incubated at −20° C. for 15 min and then centrifuged at 10,000×g for 10 min. The pellet was washed with 70% ethanol, air dried and dissolved in 7.5 μl of ddH₂O.

### Preparation of Biotin-Labeled PCRcRNA

Biotin-labeled nucleotide Bio-11-CTP and Bio-16-UTP were purchased from Enzo Biochem (Enzo Biochem, New York, N.Y.). The T7 RNA polymerase MEGAscript T7 Kit was purchased from Ambion (Ambion, Austin, Tex.). Reaction mixtures contained 2.0 μl of 10× T7 RNA polymerase buffer, 2.0 μl of 75 mM ATP, 2.0 μl of 75 mM GTP, 1.5 μl of 75 mM CTP, 1.54 μl of 75 mM UTP, 3.75 μl of 10 mM Bio-11-CTP, 3.75 μl of 10 mM Bio-16-UTP, 2.0 μl of 10× T7 RNA polymerase enzyme mix, and 1.5 μl of PCRcDNA (as prepared above). The reaction mixture was incubated at 37° C. for 5 hr. The labeled cRNA was purified with an RNeasy Mimi kit and eluted in 50 μl of ddH2O. It was fragmented at 95° C. for 35 min in a solution containing 40 mM Tris-acetate (pH 8.1), 100 mM KOAc, and 30 mM MgOAc. The fragmented cRNA was used either immediately for chip hybridization or stored in a −80° C. freezer.

### Hybridization Against Affymetrix ATH1 GeneChip®

A hybridization mixture, consisting of 50 μg of fragmented cRNA, 125 μl of 2× MES buffer (0.2 M MES pH6.7, 2M NaCl, 0.02% Triton), 6.25 μl of acetylated BSA (20 μg/μl), 2.5 μl of herring sperm DNA (10 μg/μl), 2.5 μl of biotinylated Control Oligo (5 nM), and ddH2O in a total volume of 250 μl, was heated at 95° C. for 5 min and then allowed to equilibrate at 45° C. Affymetrix Arabidopsis ATH1 Gene Chips were first treated at 45° C. for 5 min with 250 μl of prehybridization solution consisting of 125 μl of 2× MES buffer and 123 μl of ddH2O. They were then incubated with the hybridization solution at 45° C. for 16 hr in a rotary agitation hybridization oven.

### GeneChip Washing, Staining and Scanning

After the hybridization and removal of hybridization solution, the chips were washed with 6× SSPE-T (0.9M NaCl, 0.06M NaH₂PO₄ pH 6.7, 6 mM EDTA and 0.01% Triton) for ten times using the Affymetrix Fluidics Station. Chips were rinsed once with 0.1× MES and then incubated with 0.1× MES at 45° C. for 15 min in a rotary oven. The chips were rinsed with 1× MES before being stained with 220 μl of staining solution containing 205 μl of 1× MES, 23 μl of acetylated BSA (20 μg/ul), and 2.3 μl of phycoerythrin-strepavidin conjugate (1 mg/ml) (Molecular Probes, Eugene, Oreg.). Chips were incubated in staining solution at 35° C. for 15 min in a rotary oven. The chips were subsequently washed with 6× SSPE-T for ten times on Affymetrix Fluidic Station. Chips were immediately scanned on a HP GeneArray Scanner.

### GeneChip Image Quantification and Data Processing

GeneChip images were quantified and calculated by Affymetrix Microarray Suite Version 5.0.

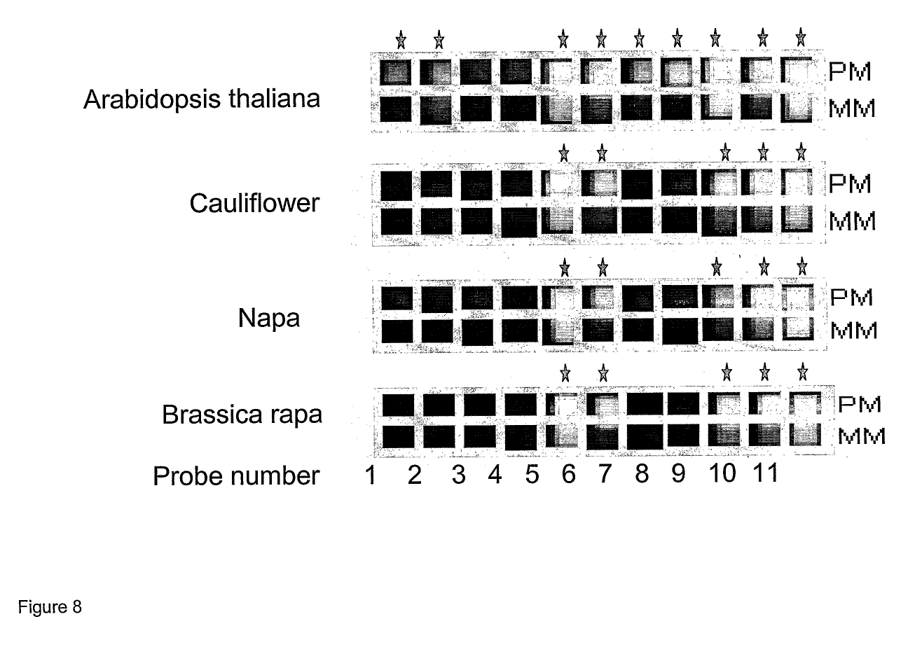

FIG. 8 is GeneChip Arobidopsis ATH1 hybridization images of chlorophyll A-B binding protein of Arabodopsis thaliana, Cauliflower, Napa and Brassica rapa. PM and MM denote perfect match probes and mismatch probes respectively. The probes that produce a relatively high hybridization signal are labeled with a star.

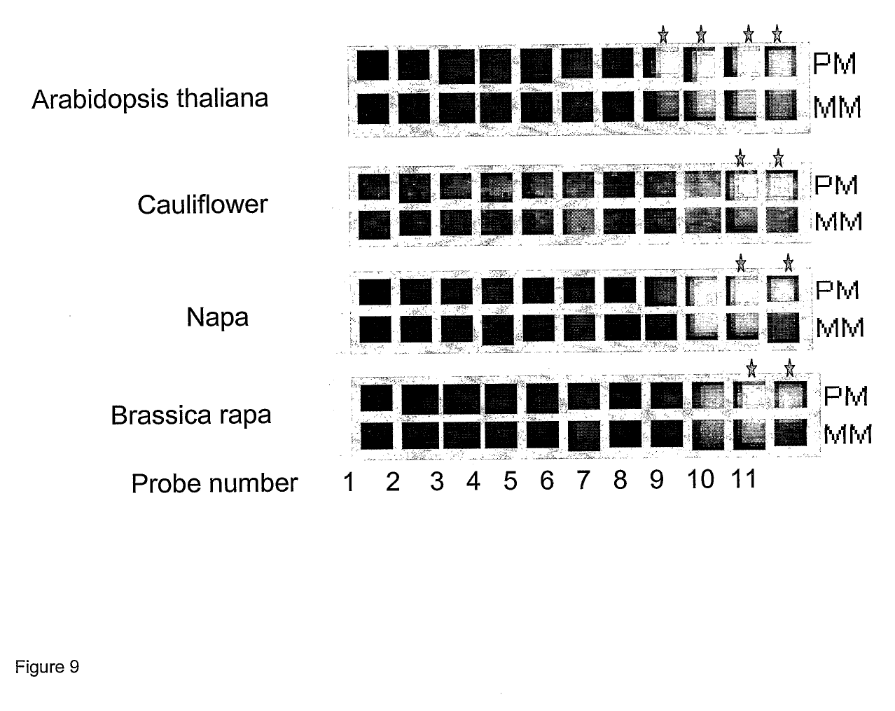

FIG. 9 is GeneChip Arobidopsis ATH1 hybridization images of mitochondrial F1-ATPase, gamma subunit of Arabodopsis thaliana, Cauliflower, Napa and Brassica rapa. PM and MM denote perfect match probes and mismatch probes respectively. The probes that produce a relatively high hybridization signal are labeled with a star. Amplification of chlorophyll A-B binding protein of Arabodopsis thaliana, Cauliflower, Napa and Brassica rapa.

From the probes that produce a relatively high hybridization signal, the one that closes to 5′ end for all four species was chosen as a forward primer for PCR amplification to maximize the length of PCR fragment. For chlorophyll A-B binding protein (Lhca2), the probe 5 is chosen (see FIG. 8). The sequence of the probe 5 of chlorophyll A-B binding protein on Arabidopsis ATH1 GeneChip was obtained from Affymetrix website (available online at affymetrix.com). It is:

| | |
|---|---|
| Lhca2-5: GGCAGTGATGGGTGCTTGGTTCCAA | (SEQ ID NO: 16) |

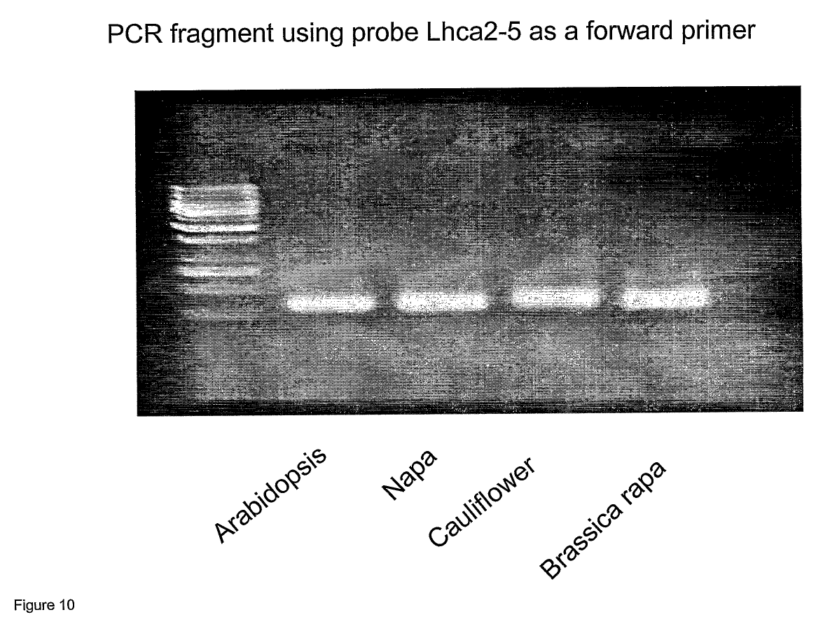

The Anchoring primer sequence is 5′CGCAGAGTGAATTAATACGACTCACT3′ (SEQ ID NO: 15). PCRcDNA of Arabodopsis thaliana, Cauliflower, Napa and Brassica rapa as described above were diluted 100 folds. PCR reaction for target gene amplification contained 10.0 μl of the PCR dilute, 5.0 μl of 10× PCR reaction buffer, 1.0 μl of 10 mM dNTPs, 1.5 μl of 10 μM anchoring primer, 1.5 μl of 10 μM Lhca2-5 primer, and 1.0 μl of Advantage Taq DNA polymerase in a volume of 50.0 μl (Clontech, Palo Alto, Calif.). The PCR amplification was performed with the following cycling parameters: 95° C. 1 min, then 30 cycles of 95° C. 15 sec and 68° C. 3 min, in a MJ Research Tetrad thermocycler (MJ Research, Waltham, Mass.). FIG. 10 is an image of agarose-gel electrophoresis of the PCR products of plant chlorophyll A-B binding protein.

Amplification of Mitochondrial F1-ATPase, Gamma Subunit of, Arabodopsis thaliana, Cauliflower, Napa and Brassica rapa

From the probes that produce a relatively high hybridization signal, the one that closes to 5′ end in all four species was chosen as one of primers for PCR amplification to maximize the length of PCR fragment. For mitochondrial F1-ATPase, gamma subunit (F1-ATPase), the probe 10 is chosen (see FIG. 9). The sequence of the probe 10 of F1-ATPase on Arabidopsis ATH1 GeneChip was obtained from Affymetrix website (available online at affymetrix.com). It is:

| | |
|---|---|
| F1-ATPase-10: | |
| ACAGGACTCGTCAAGCTTCTATTAC | (SEQ ID NO: 17) |

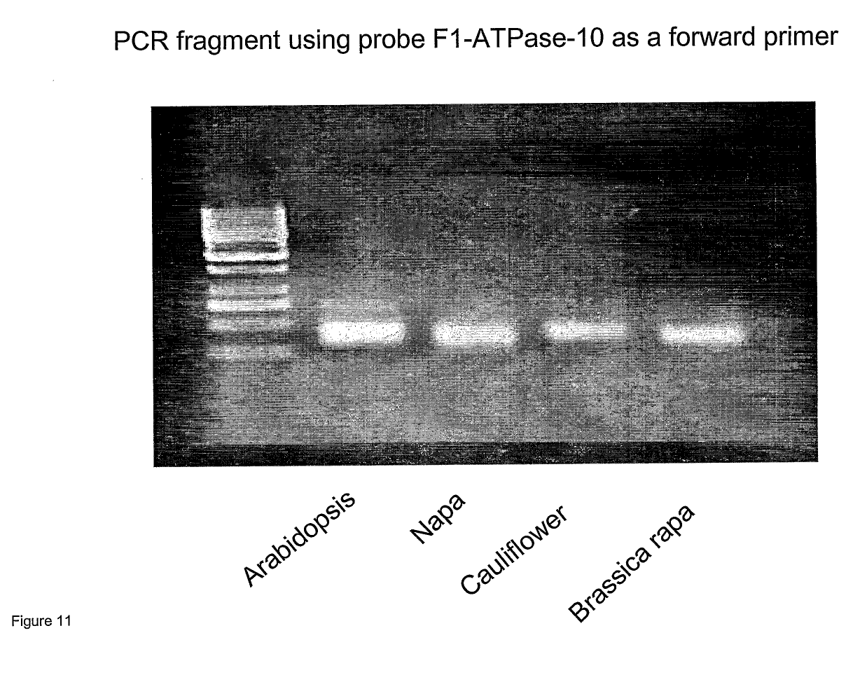

The Anchoring primer sequence is 5′CGCAGAGTGAATTAATACGACTCACT3′ (SEQ ID NO: 15). PCRcDNA of Arabodopsis thaliana, Cauliflower, Napa and Brassica rapa as described above were diluted 100 folds. PCR reaction for target gene amplification contained 10.0 μl of the PCR dilute, 5.0 μl of 10× PCR reaction buffer, 1.0 μl of 10 mM dNTPs, 1.5 μl of 10 μM anchoring primer, 1.5 μl of 10 μM F1-ATPase-10 primer, and 1.0 μl of Advantage Taq DNA polymerase in a volume of 50.0 μl (Clontech, Palo Alto, Calif.). The PCR amplification was performed with the following cycling parameters: 95° C. 1 min, then 30 cycles of 95° C. 15 sec and 68° C. 3 min, in a MJ Research Tetrad thermocycler (MJ Research, Waltham, Mass.). FIG. 11 is an image of agarose-gel electrophoresis of the PCR products of plant mitochondrial F1-ATPase, gamma subunit.

Sequencing Chlorophyll A-B Binding Protein of Arabodopsis thaliana, Cauliflower, Napa and Brassica rapa

PCR fragment of chlorophyll A-B binding protein of Arabodopsis thaliana, Cauliflower, Napa and Brassica rapa as described above was purified with QIAquick PCR purification columns (Qiagen, Chatsworth, Calif., USA) and eluted in 20.0 μl of ddH2O. Each sequencing reaction contained 30 ng of PCR fragment, 4 μl of Bigdye terminator ready solution (Applied biosystem, Foster City, Calif.), 0.5 μl of 10 μM Lhca2-5 primer in a volume of 10 μl. The sequencing reaction was performed with the following cycling parameters: 25 cycles of 95° C. 10 sec, 50° C. 5 sec, and 60° C. 4 min, in a MJ Research Tetrad thermocycler (MJ Research, Waltham, Mass.). The sequencing fragment was purified with Qiagen DyeEx spin kit (Qiagen, Chatsworth, Calif.). The elutent was dried at 70° C. for two hours, dissolved in 10 μl of deionized formamide, and loaded into ABI 3100 genetic analyzer (Applied Biosystem, Foster City, Calif.) for sequencing. Sequences were aligned by CLUSTALW (available on web site align.genome.jp).

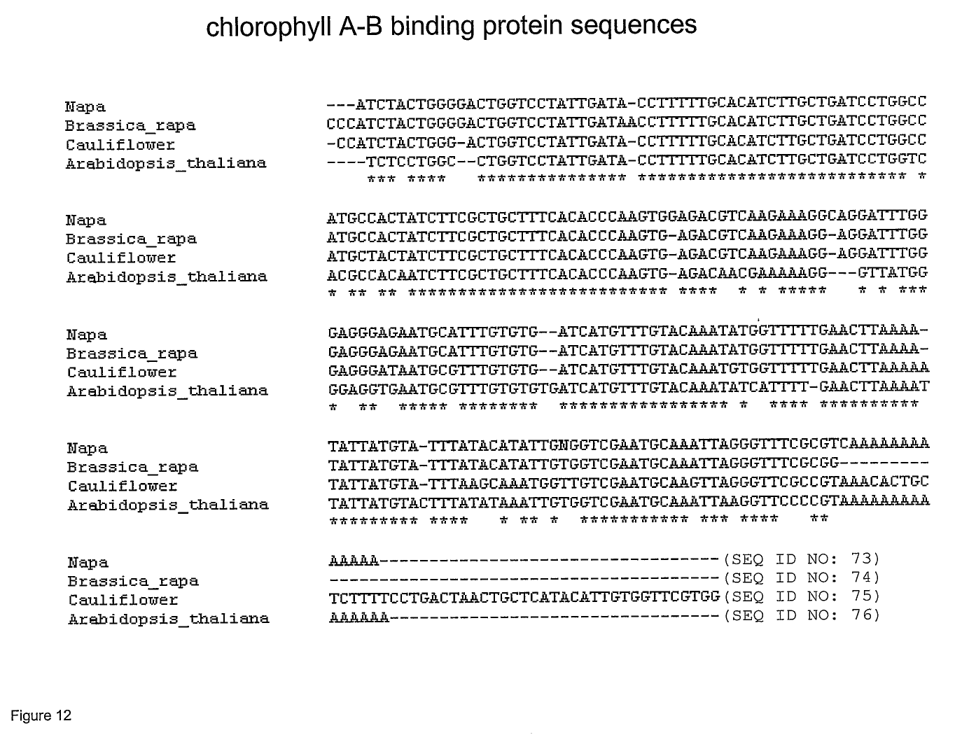

FIG. 12 shows the chlorophyll A-B binding protein sequences obtained from above sequencing reaction. All four sequences are highly homology to each other. Sequence alignment reveals highly conserved regions (marked as stars) as well as divergent regions.

Sequencing Mitochondrial F1-ATPase, Gamma Subunit of, Arabodopsis thaliana, Cauliflower, Napa and Brassica rapa

PCR fragment of F1-ATPase of Arabodopsis thaliana, Cauliflower, Napa and Brassica rapa as described above was purified with QIAquick PCR purification columns (Qiagen, Chatsworth, Calif., USA) and eluted in 20.0 μl of ddH2O. Each sequencing reaction contained 30 ng of PCR fragment, 4 μl of Bigdye terminator ready solution (Applied biosystem, Foster City, Calif.), 0.5 μl of 10 μM F1-ATPase-10 primer in a volume of 10 μl. The sequencing reaction was performed with the following cycling parameters: 25 cycles of 95° C. 10 sec, 50° C. 5 sec, and 60° C. 4 min, in a MJ Research Tetrad thermocycler (MJ Research, Waltham, Mass.). The sequencing fragment was purified with Qiagen DyeEx spin kit (Qiagen, Chatsworth, Calif.). The elutent was dried at 70° C. for two hours, dissolved in 10 μl of deionized formamide, and loaded into ABI 3100 genetic analyzer (Applied Biosystem, Foster City, Calif.) for sequencing. Sequences were aligned by CLUSTALW (available on web site http://align.genome.jp).

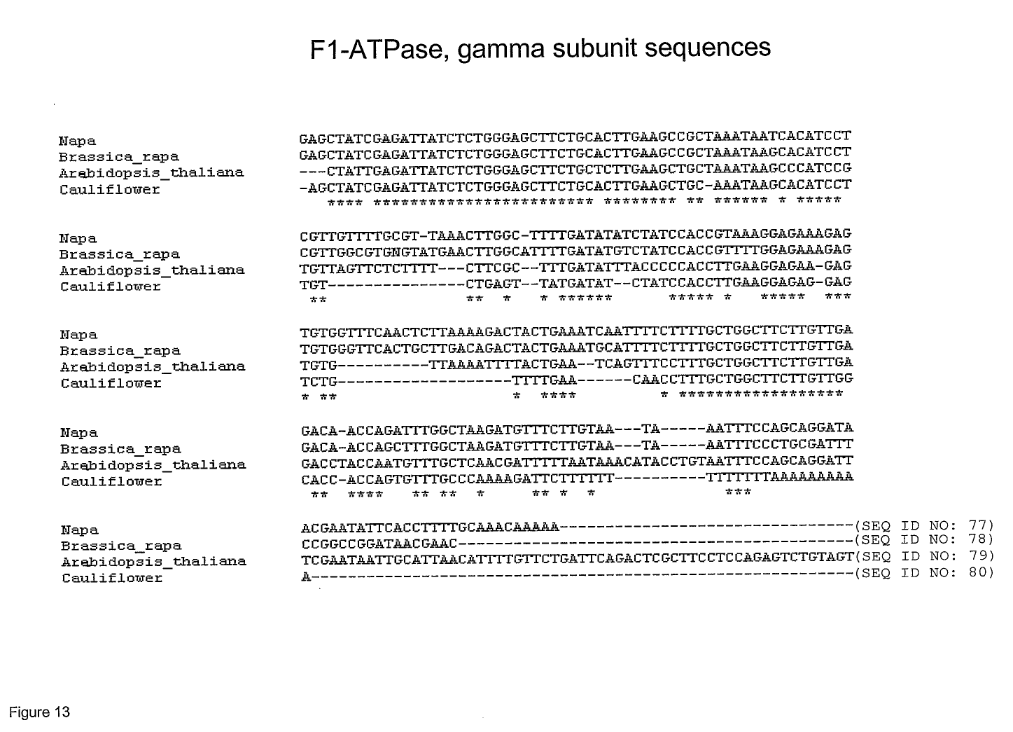

FIG. 13 shows the F1-ATPase sequences obtained from above sequencing reaction. The conserved nucleotides are marked with stars.

1-10. (canceled)

11. A method of selecting a primer suitable for PCR amplification of a heterologous nucleic acid comprising the steps of:
a) providing a nucleic acid corresponding to an expressed sequence from a species of interest;
b) hybridizing the nucleic acid to a microarray of one or more oligonucleotides derived from expressed sequences of a species different from the species of interest;
c) detecting hybridization of the nucleic acid to the one or more oligonucleotides on the microarray;
d) identifying and selecting those oligonucleotides which hybridize to the nucleic acid at a level suitable to act as PCR primers.

12. The method of claim 11, wherein the step of providing a nucleic acid comprises the steps of:
a) isolating RNA from a species of interest; and
b) synthesizing cDNA from said RNA.

13. The method of claim 11, further comprising the step of synthesizing said heterologous nucleic acid with an anchor sequence and the one or more oligonucleotides selected in step d).

14. The method of claim 12, further comprising the step of synthesizing said cDNA with an anchor sequence.

15. The method of claim 14, further comprising the step of synthesizing cRNA from said cDNA.

16. The method of step 15, further comprising the step of fragmenting said cRNA.

17. The method of claim 11, wherein the microarray comprises oligonucleotides derived from transcribed gene sequences.

18. The method of claim 17 wherein the oligonucleotides are between 8 and 60 bases in length.

19. The method of claim 17 wherein the microarray comprises oligonucleotides derived from least 1,000 expressed sequences.

20. A method for PCR amplification of a heterologous nucleic acid comprising the steps of:
a) providing a nucleic acid corresponding to an expressed gene sequence from a species of interest;
b) hybridizing the nucleic acid to a microarray of two or more oligonucleotides derived from expressed gene sequences of a species different from the species of interest;
c) detecting hybridization of the nucleic acid to the two or more oligonucleotides on the micro array;
d) identifying and selecting those oligonucleotides which have hybridized to the nucleic acid to levels suitable to act as PCR primers; and
e) amplifying the nucleic acid using one of the identified oligonucleotides or a portion thereof as a first primer to perform PCR using DNA derived from the species of interest as template.

21. The method of claim 20, wherein the step of providing a nucleic acid comprises the steps of:
a) isolating RNA from a source of interest;
b) synthesizing cDNA from said RNA.

22. The method of claim 20, further comprising using an anchor sequence as a second primer in the amplification of step e).

23. The method of claim 21, further comprising the step of synthesizing said cDNA with an anchor sequence.

24. The method of claim 21, further comprising the step of synthesizing cRNA from said cDNA.

25. The method of step 24, further comprising the step of fragmenting said cRNA.

26. The method of claim 20, wherein the microarray comprises oligonucleotides derived from transcribed gene sequences.

27. The method of claim 26 wherein the oligonucleotides are between 8 and 60 bases in length.

28. The method of claim 26 wherein the microarray comprises oligonucleotides derived from at least 1,000 different expressed sequences.

29. The method of claim 22 further comprising using said anchor sequence as a second primer in the amplification of step e).

30. A method for determining the sequence of a heterologous nucleic acid comprising the steps of:
a) providing a nucleic acid corresponding to an expressed sequence from a species of interest;
b) hybridizing the nucleic acid to a microarray of one or more oligonucleotides derived from expressed sequences of a species different from the species of interest;
c) detecting hybridization of the nucleic acid to the one or more oligonucleotides on the microarray;
d) identifying and selecting those oligonucleotides which have hybridized to the nucleic acid to levels suitable to act as PCR primers;
e) amplifying the nucleic acid using one of the identified oligonucleotides or a portion thereof as a first primer to perform PCR using DNA derived from the species of interest as template; and
f) determining the sequence of the amplified nucleic acid.

31-45. (canceled)
